(19) United States
(12) Patent Application Publication
(10) Pub. No.: US 2008/0025997 A1
(43) Pub. Date: Jan. 31, 2008

(54) Cysteine-rich region of respiratory syncytial virus and methods of use therefor

(76) Inventors: Fernando P. Polack, Bethesda, MD (US); Pablo M. Irusta, Washington, DC (US); Steven R. Kleeberger, Chapel Hill, NC (US)

Correspondence Address: EDWARDS ANGELL PALMER & DODGE LLP;Client: JHU, P.O. BOX 55874, BOSTON, MA, 02205, US

(21) Appl. No.: 11/641,625

(22) Filed: Dec. 18, 2006

Related U.S. Application Data
(63) Continuation-in-part of application No. PCT/US05/21538, filed on Jun. 16, 2005.
(60) Provisional application No. 60/580,167, filed on Jun. 16, 2004.
(60) Provisional application No. 60/685,058, filed on May 26, 2005.

(51) Int. Cl. C07K 14/135; A61K 31/7088; A61K 38/14; A61K 39/155; C12N 15/45; C12Q 1/02; G01N 33/53; G01N 33/68; C12Q 1/68; C12N 15/86; C07K 9/00; A61K 48/00; A61P 37/00

(52) U.S. Cl. 424/186.1; 424/93.2; 435/29; 435/320.1; 435/6; 436/86; 514/44; 514/8; 530/322; 530/395; 536/23.72

(57) ABSTRACT

The invention features methods and compositions featuring an RSV Glycoprotein fragment for modulating an immune response in a subject.

### REFERENCE TO RELATED APPLICATIONS

This application is a continuation-in-part application of International Application No. PCT/US05/21538, which was filed, Jun. 16, 2005, which claims benefit of U.S. Provisional Application Ser. No. 60/580,167, filed on Jun. 16, 2004 and U.S. Provisional Application Ser. No. 60/685,058 filed on May 26, 2005, the contents each of which are incorporated herein by reference.

### BACKGROUND OF THE INVENTION

The immune system plays a critical role in the resolution of a variety of diseases. The immune system protects the body from potentially harmful substances by recognizing and responding to antigens. During pathogen infection, for example, the immune system relies on pattern-recognition receptors that allow the immune system to generate an immediate response. This type of immune response is an innate immune response. In contrast to innate immunity, adaptive immunity develops when the body is exposed to various antigens and builds a defense that is specific to that antigen.

Immune system disorders occur when the immune response is inappropriate, excessive, or lacking. Allergies involve an immune response to a substance that, in the majority of people, the body perceives as harmless. Transplant rejection involves the destruction of transplanted tissues or organs and is a major complication of organ transplantation. Blood transfusion reaction is a complication of blood administration. Autoimmune disorders (such as systemic lupus erythematosus and rheumatoid arthritis) occur when the immune system acts to destroy normal body tissues. Immunodeficiency disorders (such as inherited immunodeficiency and AIDS) occur when there is a failure in all or part of the immune system.

The Toll-like receptor 4 (TLR4) functions in innate immunity. TLR4 activates innate inflammation by promoting nuclear translocation of the NF-κB transcription factor through a conserved signal transduction pathway. NF-κB induces production of inflammatory cytokines, chemokines, vasoactive agents, adhesion molecules, proteases and antiproteases involved in host defense. Activation of TLR4 can be elicited by endotoxin (LPS) and its effects are associated with a variety of illnesses, ranging from gram-negative sepsis to asthma. Respiratory syncytial virus (RSV) also can activate TLR4 through interaction with the viral fusion (F) glycoprotein. While host immunity clearly is important for restricting and resolving RSV infection, it also is thought to contribute to RSV disease. Increased understanding of the mechanisms that limit a host immune response to RSV would have wide applicability to a variety of diseases and disorders associated with an inappropriate or exaggerated immune response. Improved therapeutic methods for modulating innate immunity are urgently needed for the treatment of diseases and disorders associated with an exaggerated immune response (e.g., inflammatory disorder, rejection of a transplanted organ, sepsis). Improved therapeutic methods for the treatment of diseases or disorders that require the enhancement of an adaptive immune response, such as pathogen infections and neoplasia are also required.

### SUMMARY OF THE INVENTION

The invention features methods and compositions featuring an RSV Glycoprotein fragment for modulating an immune response in a subject.

In one aspect, the invention generally features an isolated RSV Glycoprotein fragment (e.g., a Glycoprotein cysteine rich region (GCRR) that includes amino acids 164-189 of the RSV Glycoprotein, or at least amino acids 173-186 of an RSV Glycoprotein having immunomodulatory activity).

In other aspects, the invention features isolated RSV Glycoprotein nucleic acid molecules encoding the RSV Glycoprotein, vectors containing the nucleic acid molecules, and host cells containing those vectors. In various embodiments, the vector is an expression vector. In another embodiment, the RSV Glycoprotein nucleic acid molecule is positioned for expression. In another embodiment, RSV Glycoprotein nucleic acid molecule is operably linked to a promoter. In another embodiment, the promoter is suitable for expression in a mammalian cell. In yet another embodiments, the vector comprises a second polynucleotide sequence encoding an antigenic polypeptide of interest position for expression in a mammalian cell.

In another aspect, the invention features a viral vector containing an RSV Glycoprotein nucleic acid molecule encoding a polypeptide of a previous aspect. In one embodiment, the viral vector contains an inactivating mutation. In other embodiments, the viral vector is replication competent or replication incompetent. In yet other embodiments, the viral vector is selected from the group consisting of adenoviral vectors, adeno-associated viral vectors, retroviral vectors, lentiviral vectors, alphaviral vectors, and herpes virus vectors. In still other embodiments, the vector comprises a second polynucleotide sequence encoding an antigenic polypeptide of interest.

In another aspect, the invention features a host cell (e.g., a mammalian cell or a human cell) containing the viral vector of any previous aspect. In other embodiments, the cell expresses an RSV Glycoprotein fragment. In one embodiment, the cell may be in vitro or in vivo. Preferably, the host cell expresses an RSV Glycoprotein fragment at a level sufficient to modulate an immune response in an organism containing the host cell.

In yet another aspect, the invention features a composition containing an effective amount of an RSV Glycoprotein fragment in a pharmaceutically acceptable excipient, where the fragment is capable of modulating an immune response in a subject.

In yet another aspect, the invention features a pharmaceutical composition containing an effective amount of a nucleic acid molecule encoding an RSV Glycoprotein fragment of any previous aspect in a pharmaceutically acceptable excipient, where the fragment is capable of modulating an immune response in a subject.

In yet another aspect, the invention features a pharmaceutical composition containing an effective amount of a vector containing a nucleic acid molecule encoding an RSV Glycoprotein fragment of a previous aspect in a pharmaceutically acceptable excipient, where the fragment is capable of modulating an immune response in a subject. In one embodiment, the RSV Glycoprotein nucleic acid molecule is positioned for expression in a mammalian cell (e.g., a cell in vitro or in vivo). In another embodiment, the vector further contains a second nucleic acid molecule encoding an antigen of interest (e.g., a tumor antigen or pathogen antigen).

In another aspect, the invention features a pharmaceutical composition containing an effective amount of a viral vector of a previous aspect.

In yet another aspect, the invention features an immunogenic composition containing an RSV Glycoprotein fragment in a pharmaceutically acceptable excipient. In one embodiment, the immunogenic composition further contains an antigen of interest. In another embodiment, the RSV Glycoprotein fragment enhances an immune response against the antigen of interest.

In another aspect, the invention features method of modulating an immune response in a subject in need thereof, the method comprising administering to the subject an RSV Glycoprotein fragment capable of modulating an immune response or a polynucleotide encoding the fragment.

In another aspect, the invention features a method of decreasing a Toll-like receptor (TLR) function in a subject in need thereof. The method involves administering to the subject (e.g., a mammal, such as a human) an RSV Glycoprotein fragment capable of modulating an immune response or a polynucleotide encoding the fragment. In other embodiments, the TLR is selected from the group consisting of TLRs 1-11 (e.g., TLR2, TLR4, or TLR9).

In another aspect, the invention features a method of decreasing an inflammatory response in a subject (e.g., a mammal, such as a human) in need thereof, the method comprising administering to the subject an RSV Glycoprotein fragment capable of modulating an immune response or a polynucleotide encoding the fragment. In one embodiment, the method stabilizes, reduces the symptoms of, or ameliorates a disease or disorder characterized by an increase in Toll-like receptor signaling. In another embodiment, the immune response is an adverse immune response selected from the group consisting of an autoimmune disorder, an inflammatory disorder, rejection of a transplanted organ, and sepsis. In another embodiment, the disease or disorder is a pathogen infection or a neoplasia. In another aspect, the invention features a method of enhancing an immune response in a subject (e.g., a mammal, such as a human) against an immunogenic composition. The method involves administering an effective amount of a pharmaceutical composition containing an RSV Glycoprotein fragment of a previous aspect or a polynucleotide encoding the fragment to a subject before, during, or after the administration of an immunogenic composition, such that the subjects immune response is enhanced. In one embodiment, the immune response is an adaptive immune response. In another embodiment, the method enhances an immune response against a pathogen infection (e.g., herpes, cytomegalovirus, HIV, AIDs, influenza, malaria, or a parasite infection). In another embodiment, the method enhances an immune response against a neoplasia (e.g., melanoma).

In another aspect, the invention features a method for identifying a candidate compound that modulates an immune response in a subject (e.g., a mammal, such as a human). The method involves a) providing a cell expressing an RSV Glycoprotein nucleic acid molecule; (b) contacting the cell with a candidate compound; and (c) comparing the expression of the nucleic acid molecule in the cell contacted with the candidate compound with the expression of the nucleic acid molecule in a control cell not contacted with the candidate compound, where an alteration in the expression identifies the candidate compound as a candidate compound that modulates an immune response.

In yet another aspect, the invention provides a method for identifying a candidate compound that modulates an immune response in a subject. The method involves (a) providing a cell expressing a RSV Glycoprotein; (b) contacting the cell with a candidate compound; and (c) comparing the biological activity of the RSV Glycoprotein in the cell contacted with the candidate compound to a control cell not contacted with the candidate compound, where an alteration in the biological activity of the RSV Glycoprotein identifies the candidate compound as a candidate compound that modulates an immune response in a subject.

In various embodiments of the previous aspects, the cell is a mammalian cell. In other embodiments, the biological activity is monitored with an enzymatic assay, an immunological assay, detecting cytokine release, or by detecting NFκB level or localization.

In yet another aspect, the invention features method for identifying a candidate compound that modulates an immune response in a subject. The method involves a) contacting a RSV Glycoprotein with a candidate compound; and (b) detecting binding of the candidate compound to the RSV Glycoprotein, where the binding identifies the candidate compound as a candidate compound that modulates an immune response in a subject. In yet another aspect, the invention features a method for enhancing an immunomodulatory activity of an RSV Glycoprotein. The method involves a) introducing an alteration in a naturally occurring RSV Glycoprotein amino acid sequence; and b) detecting an alteration in the immunomodulatory activity of the RSV Glycoprotein.

In yet another aspect, the alteration is detected by assaying cytokine release, by assaying NFκB level or localization, by assaying Toll-like receptor signaling. In another embodiment, the Toll-like receptor is selected from the group consisting of TLRs 1-11 (e.g., TLR2, TLR4, or TLR9). In another embodiment, the alteration is a change in the amino acid sequence (e.g., an insertion, deletion, nonsense mutation, or missense mutation). In another embodiment, the alteration is the replacement of a natural amino acid with an unnatural amino acid or amino acid analog.

In a final aspect, the invention provides a method for selecting an RSV Glycoprotein nucleic acid molecule having improved immunomodulatory activity. The method involves a) introducing an alteration in a naturally occurring RSV Glycoprotein nucleic acid sequence; and b) detecting an alteration in the immunomodulatory activity of the encoded RSV Glycoprotein.

In various embodiments of any of the above aspects, the fragment contains cysteines at an amino acid position corresponding to cysteines 182 and 186 of human RSV or at least four cysteine residues corresponding to cysteines 173, 176, 182, and 186. In other embodiments, the fragment contains at least a Glycoprotein cysteine rich region (GCRR), at least amino acids 164-189 of the RSV Glycoprotein, or at least amino acids 173-186 of an RSV Glycoprotein. In yet other embodiments, the fragment comprises at least 10, 11, 12, 13, 14, 15, 16, 17, 18, 19, or 20 amino acids of an RSV Glycoprotein (e.g., human, bovine or ovine RSV). In another embodiment, the fragment consists essentially of a GCRR motif. In other embodiments the fragment is a fusion protein, is linked to a detectable amino acid sequence, is linked to an affinity tag.

In various embodiments of any of the above aspects, the immune response is an innate immune response, an adaptive immune response, a cytotoxic T cell response, or cytokine release.

In other embodiments of any of the above aspects, the immune response is an adverse immune response selected from the group consisting of an autoimmune disorder, an inflammatory disorder, rejection of a transplanted organ, and sepsis. In yet other embodiments of the previous aspects, the RSV Glycoprotein fragment is provided to the subject by inhalation. In other embodiments of the previous aspects, the RSV Glycoprotein fragment is administered to the lung epithelium.

### BRIEF DESCRIPTION OF THE DRAWINGS

FIG. 1 is a schematic diagram showing the structure of RSV Glycoprotein based on an RSV A2 strain. The schematic shows the relative positions of the RSV Glycoprotein cysteine rich region (GCRR), a CX₃C motif, disulfide bridges, and 13 amino acid segment in the RSV Glycoprotein. Disulfide bridges within the GCRR are represented by dashed lines between the relevant cysteines.

FIGS. 2A-2B are graphs showing monocyte production of interleukin-6 or IL-1β after stimulation with purified protein (FIG. 2A) or live RSV (FIG. 2B). Purified human monocytes were stimulated with the following proteins (FIG. 2A): RSV F protein (closed bar), RSV Glycoprotein from antigenic subgroup A (GA protein, open bar) or antigenic subgroup B (G_{B} protein, gray bar), RSV F and G_{A} proteins (horizontal striped bar), RSV F and G_{B} proteins (vertical striped bar), RSV F protein and GCRR peptide (3 μg, dotted bar and 15 μg, crossed bar), RSV F protein and bovine serum albumin (dark gray bar), and RSV F protein and Hep-2 cell lysate (black stripped bar). FIG. 1B shows monocyte dose-response curves to RSV (closed squares) or the ΔG mutant (open squares). IL-6 and IL-1β were measured by immunoassay in supernatant fluids eighteen hours after incubation for both

FIGS. 2A and 2B. Results are mean ±SEM and are representative of three independent experiments.

FIGS. 3A-3B are graphs showing monocyte production of interleukin-6 after incubation with viruses. Purified monocytes were incubated with the following viruses (FIG. 3A): incubated with RSV (closed bar), Vero cell lysate (gray bar), ΔG (open bar), mG (horizontal striped bar), mG with soluble G (black stripped bar), G_{Δ172-187} (gray horizontal stripped bar), or ΔG+G¹⁶⁴⁻¹⁷⁶ peptide (10 μg) (dotted bar). All viruses were inoculated at 10⁵ pfu (MOI=1). In FIG. 3B monocytes were incubated with UV-inactivated RSV (closed bar) or ΔG (open bar); In FIG. 3C monocytes were incubated with RSV lacking one of four cysteines in the GCRR and therefore the corresponding disulfide bridge (G_{Cys182Arg} and G_{Cys186Arg}), in addition to the correspondent control virus to examine cytokine production. IL-6 was measured by immunoassay in supernatant fluids eighteen hours after infection. Results are mean ±SEM and are representative of two-three independent experiments.

FIGS. 4A-4C shows that RSV Glycoprotein, through its GCRR, inhibits nuclear translocation of NF-κB. FIGS. 4A and 4B are graphs showing nuclear translocation of transcription factor NF-κB in human monocytes. Monocytes were stimulated for 60 min with (FIG. 4A) F and G protein individually and in combination or (FIG. 4B) RSV or ΔG (MOI=1), and NF-κB subunits p50 and p65 detected in purified nuclei by a modified immunoassay. FIG. 4C is a Western blot showing the effect of stimulating monocytes for 60 minutes with RSV, ΔG and G_{Δ172-187} (MOI=1), on cytoplasmatic IκBα.

FIGS. 5A-5F shows the role of RSV Glycoprotein during infection in vivo. FIGS. 5A-5E are graphs showing the effects of stimulating purified murine alveolar macrophages with RSV proteins; in FIG. 5A the following proteins were used: RSV F protein (closed bar), RSV Glycoprotein from antigenic subgroup A (G_{A} protein, open bar) or RSV F and Glycoprotein_{A} (horizontal stripped bar); and in FIG. 5B, the following proteins were used: RSV (dark gray bar), ΔG (vertical stripped bar) or G_{Δ172-187} (black horizontal stripped bar). FIG. 5C shows the effect of virus titration in lungs of mice four days after infection. Viruses were inoculated intransally at 10⁶ pfu (n=5/group); FIG. 5D shows intracellular expression of IL-6 by alveolar macrophages from mice infected with RSV, ΔG or placebo analyzed by flow cytometry 24 hours. post-infection. Viruses were inoculated intranasally at 10⁶ pfu. FIG. 5E is a graph showing the pulmonary histopathology score assessing PMN and macrophage infiltration after infection with RSV (closed squares), ΔG (open squares) or mG (gray squares). FIG. 5F is a series of photomicrographs showing pulmonary histopathology in mice 24 hours after infection with the indicated virus (PAS, 10×).

FIGS. 6A and 6B are graphs showing the production of interleukin-6 and interleukin 1-beta by monocytes after stimulation with endotoxin. Cells were incubated with LPS (1 μg; dosed bar), LPS and GCRR peptide (3 μg, dotted bar and 15 μg, crossed bar), and LPS and control human immunodeficiency virus V3 loop peptide (3 μg, horizontal stripped bar and 15 μg, vertical stripped bar) or GSRR peptide lacking the four cysteines and disulfide bridges (open bar). Supernatants were collected eighteen hours after addition of stimulants. IL-6 and IL-1β were measured by immunoassay. Results are mean ±SEM and are representative of three independent experiments.

FIGS. 7A and 7B are graphs showing modulation of TLR2- and TLR9-mediated inflammatory responses. In FIG. 7A purified human monocytes were stimulated with: PGN or PGN+Glycoprotein; and in FIG. 7B with CpG DNA or CpG DNA. Supernatants were collected 18 hours after addition of stimulants. IL-10 was measured by immunoassay. Results are mean ±SEM.

FIGS. 8A-8C are graphs showing the quantification of RSV and M2-specific CD8⁺ T cells during infection with RSV or recombinant G-deficient viruses. FIG. 8A shows the effect of infection on PMC that were isolated at different time points post-infection, stained with anti-CD8 antibodies and the M2 tetramer and analyzed by flow cytometry. FIG. 8C shows the effect of the M2⁸²⁻⁹⁰ peptide on PMC that were subsequently stained for CD8 and IFN-γ and analyzed by flow cytometry. FIG. 8C shows the number of IFN-γ producing cells was determined using an immunospot assay. PMC were isolated at the peak of CTL response on day 9 were stimulated with the M2⁸²⁻⁹⁰ peptide-loaded A-20 target cells and stained for IFN-γ. Open bars: RSV, dotted bars: ΔG, and stripped bars: mG. Results are mean ±SEM and representative of 2-3 independent experiments.

FIGS. 9A and 9B are graphs showing RSV-specific cytolytic responses after infection with RSV or recombinant G-deficient viruses. PMC were stimulated with (FIG. 9A) RSV-infected or (FIG. 9B) M282-90-peptide-loaded A-20 target cells for 6 hours, and cytolytic activity was tested using the appropriate target cells with a 50:1 effector to target ratio using a LDH-release assay. Open squares: RSV, stripped triangles: mG and dotted diamonds: ΔG. Results are mean ±SEM and representative of 2 independent experiments.

FIGS. 10A and 10 B are graphs showing the quantification of RSV-specific CD8+ T cells after infection with wild-type RSV, mG or co-infection with wG. FIGS. 10A and 10B show the quantitation of an immunospot assay where PMC were isolated on day 7 after infection, stimulated with M282-90 peptide-loaded A-20 target cells, stained for IFN-γ. Results are expressed as mean ±SEM.

FIGS. 11A and 11B show the effect of RSV infection on viral lung titers and pulmonary histopathology. FIG. 11A is a graph showing viral titers in lungs after infection with wild-type RSV or recombinant viruses lacking one or both forms of RSV Glycoprotein on day 4 after infection. In FIG. 11A, open squares: RSV, stripped triangles: mG, and dotted diamonds: ΔG. FIG. 11B shows pulmonary histopathology in BALB/c mice seven days after infection with RSV or G-deficient viruses (periodic acid schiff, 10×).

FIGS. 12A and 12B are graphs showing the quantification of the RSV-specific CD8+ T cells after infection with wild-type RSV or the ΔG172.187 virus. In FIG. 12A PMC were isolated from infected mice on day 10 post-infection, stimulated with the M282-90 peptide-loaded A-20 target cells, and the numbers of cells producing IFN-γ were determined by an immunospot assay. The results are expressed as mean ±SEM. FIG. 12B shows viral titers in lungs on day 4 after infection of BALB/c mice with wild-type RSV or ΔG1712-187 virus.

FIG. 13 provides a sequence alignment of HRSV-G, type A (159-186), HRSV-G, type B (159-186) and 55 kD TNFr human (139-166). Shaded amino acid sequences are likely to be functionally significant. Alterations in the amino acid sequence of the GCRR may be made to enhance the biological activity of the RSV Glycoprotein fragment.

FIG. 14 is a graph showing the quantification of cytotoxic T lymphocyte response in mice immunized with malaria alone (Mal) as shown by the light grey bar, malaria+RSV glycoprotein cysteine rich region (amino acids 173-186) (Mal+G) as shown by the dark grey bar, or malaria+control (Mal+cont) as shown by white bar. The number of cytotoxic T lymphocytes was determined by an immunospot assay. The term “sin A20” refers to a negative control condition where the experiment was carried out in the absence of antigen presenting cells.

FIGS. 15A and 15B are flow cytometric analyses of cell lytic activity determined by granzyme-B expression in control cells infected with influenza alone (FIG. 15A) and cells infected with influenza virus and RSV-G (FIG. 15B).

### DETAILED DESCRIPTION OF THE INVENTION

### Definitions

By an “RSV Glycoprotein fragment” is meant a portion of an RSV Glycoprotein that includes the Glycoprotein cysteine rich region (GCRR) and has immunomodulatory activity. The sequence of human RSV Glycoprotein is described in Langedijk et al. Virology 243, 293-302 (1998). A sequence for human RSV Glycoprotein is available at GenBank Accession No. AF013254. Other RSV Glycoprotein fragments useful in the methods of the invention are fragments of RSV Glycoprotein subgroup A, subgroup B, bovine RSV, and ovine RSV.

By “RSV Glycoprotein nucleic acid molecule” is meant a nucleic acid molecule that encodes an RSV Glycoprotein or biologically active fragment thereof.

By “RSV Glycoprotein biological activity” is meant the ability to modulate an immune response. In one embodiment, an RSV Glycoprotein or fragment thereof reduces an innate immune response. In another embodiment, an RSV Glycoprotein enhances an adaptive immune response, such as the cytotoxic T cell response.

By “Glycoprotein cysteine rich region” is meant amino acids amino acids 173-186 of the human RSV Glycoprotein.

By “Glycoprotein central region segment” is meant amino acids 164-189 of the human RSV Glycoprotein.

By “adaptive immune response” is meant an immune response that requires prior exposure to an antigen.

An “adverse immune response” refers to any immune response having a detrimental health effect in a subject, such as inflammation. Inflammation can be caused, for example, by pathogenic infection, irritation or disease. Inflammation can also be caused by autoimmunity, wherein a subject's own antibodies react with host tissue or in which immune effector T cells are autoreactive to endogenous self-peptides and cause destruction of tissue.

“Accumulation” of inflammatory cells refers to the build up of inflammatory cells during an immune response.

By “ameliorate” is meant decrease, suppress, attenuate, diminish, arrest, or stabilize the development or progression of a disease.

A “cytokine” is a generic term for extracellular proteins or peptides that mediate cell-cell communication, often with the effect of altering the activation state of cells.

A “chemokine” is a specific type of cytokine with a conserved cysteine motif and which can serve as an attractant.

By “disease” is meant any condition or disorder that damages or interferes with the normal function of a cell, tissue, or organ.

By “fragment” is meant a portion of a protein or nucleic acid that is substantially identical to a reference protein or nucleic acid. In some embodiments the portion retains at least 50%, 75%, or 80%, or more preferably 90%, 95%, or even 99% of the biological activity of the reference protein or nucleic acid described herein. In other embodiments, the fragment comprises at least 5, 10, 11, 12, 13, 14, 15, 16, 17, 18, 19, or 20 amino acids of a reference protein or is a nucleic acid molecule encoding such a fragment.

By “immunomodulatory activity” is meant an increase or decrease in an immune response (e.g., an innate or adaptive immune response).

The term “inflamed tissue” can be used to describe any biological tissue that has mounted an immune response causing inflammation throughout or in a portion of the tissue.

By “innate immune response” is meant an immune response that does not require prior exposure to an antigen.

By “isolated nucleic acid molecule” is meant a nucleic acid (e.g., a DNA) that is free of the genes that, in the naturally occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

An “inflammatory cell” is a cell contributing to an immune response that can include, but is not limited to, follicular dendritic cells, Langerhans cells, interstitial dendritic cells, interdigitating dendritic cells, blood and veiled dendritic cells, leukocytes, lymphocytes (B-lymphocytes and T-lymphocytes), monocytes, macrophages, foam cells, tissue-specific macrophages such as alveolar macrophages, microglia, mesangial cells, histiocytes, and Kupffer cells, neutrophils, basophils, mast cells, natural killer cells, eosinophils, and polymorphonuclear cells (e.g., granulocytes). The vector may also replicate in a smooth muscle cell.

The term “immune response” refers to the process whereby inflammatory cells are recruited from the blood to lymphoid as well as non-lymphoid tissues via a multifactoral process that involves distinct adhesive and activation steps. Inflammatory conditions cause the release of chemokines and other factors that, by upregulating and activating adhesion molecules on inflammatory cells, promote adhesion, morphological changes, and extravasation concurrent with chemotaxis through the tissues.

By “modulation” is meant any alteration (e.g., increase or decrease) in a biological function or activity.

By “neoplasm” is meant a disease that is caused by or results in inappropriately high levels of cell division, inappropriately low levels of apoptosis, or both. Cancer is an example of a neoplasm.

By “polypeptide” is meant any chain of amino acids, regardless of length or post-translational modification.

By “subject” is meant a mammal, such as a human patient or an animal (e.g., a rodent, bovine, equine, porcine, ovine, canine, feline, or other domestic mammal).

By “Toll-like receptor” is meant any receptor having at least 85% amino acid sequence identity to a Toll-like receptor described herein. Exemplary Toll-like receptors include, but are not limited TLR 1-11. In particular, TLR2, TLR4, and TLR9.

By “Toll-like receptor function” is meant function in an immune response. Exemplary Toll-like receptor functions include pathogen recognition and signal transduction pathway activation.

A “therapeutically effective amount” is an amount sufficient to effect a beneficial or desired clinical result.

By “treat” is meant stabilize, reduce, or ameliorate the symptoms of any disease or disorder.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

### METHODS OF THE INVENTION

The invention provides methods and compositions featuring an RSV Glycoprotein fragment for modulating an immune response in a subject. The invention is based, in part, on the discovery that an RSV Glycoprotein or fragment thereof, has immunomodulatory activity. Specifically, an RSV Glycoprotein or fragment thereof comprising a Glycoprotein Cystein Rich Region (GCRR) is capable of inhibiting an innate immune response and enhancing an adaptive immune response. In various embodiments, the invention provides methods for preventing or treating a disease or disorder characterized by an adverse immune response, such as an autoimmune disorder, an inflammatory disorder, rejection of a transplanted organ, or sepsis. In other embodiments, the invention provides methods for the treatment of diseases or disorder that require the enhancement of an adaptive immune response, such as a pathogen infection, herpes infection, cytomegaloviral infection, bacterial infection, human immunodeficiency infection, or neoplasia (e.g., melanoma, lung, or breast cancer). In particular, the invention provides methods of providing an RSV Glycoprotein fragment to a subject to treat or prevent a disease or disorder characterized by an adverse immune response, such as an autoimmune disorder, an inflammatory disorder, rejection of a transplanted organ, or sepsis. In other embodiments, the invention provides methods for the treatment of diseases or disorder that require the enhancement of an adaptive immune response, such as a pathogen infection or a neoplasia. In yet other embodiments, the method provides methods for treating or preventing an RSV infection, influenza infection, or malaria infection in a human subject by administering at least a fragment of an RSV glycoprotein or a nucleic acid molecule encoding the RSV glycoprotein to the subject.

Inflammatory Disorders

Methods of the invention are useful for decreasing an adverse immune response, such as an inflammatory response, an autoimmune response, or the rejection of a transplanted cell, tissue, or organ. The inflammatory response can be attributed to various diseases and conditions that affect one or more organs or organ systems including, but not limited to, the peripheral nervous system, the central nervous system, skin, appendix, GI tract (including but not limited to esophagus, duodenum, and colon), respiratory/pulmonary system (including but not limited to lung, nose, pharynx, larynx), eye, genito-reproductive system, gums, liver/biliary ductal system, renal system (including but not limited to kidneys, urinary tract, bladder), connective tissue (including but not limited to joints, cartilage), cardiovascular system, muscle, breast, lymphatic system, ear, endocrine/exocrine system (including but not limited to lacrimal glands, salivary glands, thyroid gland, pancreas), and bone/skeletal system. The immune response can be an inflammatory response associated with wound formation in any tissue, including but not limited to those mentioned herein.

Inflammatory diseases that affect the peripheral nervous system include, but are not limited to, radiculitis. Inflammatory diseases of the central nervous system include acute hemorrhagic leukoencephalitis, cholesterol granuloma, meningoencephalitis, optic neuritis, and Parsonage-Aldren-Turner syndrome, but are not limited to these diseases. Inflammatory diseases of the skin can include, but are not limited to, acute infantile hemorrhagic edema, contact dermatitis, Favre-Racouchot syndrome, folliculitis, panniculitis, Riehl's melanosis, Stevens-Johnson syndrome, and trichostasis spinulosa. Inflammatory diseases of the appendix include appendicitis.

Atrophic gastritis, Barrett's esophagus, Celiac disease, colitis, colonic diverticulitis, Curling's ulcers, Cushing's ulcers, esophagitis, phlegmonous gastritis, proctitis, toxic megacolon, and typhlitis are some inflammatory diseases that affect the GI tract. Inflammatory diseases of the respiratory/pulmonary system include, but are not limited to atopic rhinitis, bronchiolitis obliterans organizing pneumonitis, pleural empyema, endogenous lipoid pneumonia, laryngeal granuloma, lymphocytic interstitial pneumonia, pharyngitis, pleuritis, sinusistis, and sterile pneumonitis. Inflammatory diseases of the eye can be blepharitis, dacryocystitis, endophthalmitis, Fuch's heterochromic cyclitis, giant papillary conjunctivitis, optic neuritis, phlyctenular keratoconjunctivitis, scleritis, but are not limited to these examples.

Diseases characterized by inflammation that affect the genito-reproductive system include, but are not limited to Bowenoid papulosis, cervicitis, cystitis, epidydymo-orchitis, peritonitis, and posthitis. Inflammatory diseases that affect the gums include cancrum oris, giant cell granuloma, gingivitis, pericoronitis, periodontitis, and pulpitis, but are not limited to these examples. Diseases states that are characterized by inflammation and that affect the liver/biliary ductal system include, but are not limited to, cholangitis and perihepatitis. Inflammatory diseases of the renal system can include chronic interstitial nephritis, Hunner's ulcer, post-streptococcal glomerulonephritis, and xanthogranulomatous pyelonephritis. Disease states that affect connective tissue include, but are not limited to, De Quervain's tenosynovitis, pyrophosphate arthropathy, reactive arthropathy, sacroilitis, synovitis, tenosynovitis, Tietze's costochondritis, and urate crystal arthropathy.

Disease states characterized by inflammation of the cardiovascular system include endocarditis, pericarditis, thrombophlebitis, and vasculitis, but are not limited to these examples. Inflammatory disease states that affect muscle include but are not limited to, myositis and Parsonage-Aldren-Turner syndrome. Mastitis and Mondor's disease of the breast are some inflammatory conditions that affect the breast. Diseases of the lymphatic system that are characterized by inflammation include mesenteric adenitis and pseudolymphoma, but are not limited to these examples. Inflammatory diseases of the ear can include diseases such as myringitis bullosa. Inflammatory diseases of the endocrine/exocrine system can include necrotizing sialometaplasia, pancreatitis, parotitis, and thyroiditis, while diseases of the bone/skeletal system characterized by inflammation include osteitis, osteitis fibrosa cystica, osteitis pubis, and periostitis, but are not limited to these examples. It is evident that many inflammatory diseases can be systemic and affect more than one organ system. Some systemic inflammatory diseases can include gangrene, Jarisch-Herxheimer reaction, and Reiter's syndrome.

Autoimmune disease is a class of diseases in which a subject's own antibodies react with host tissue or in which immune effector T cells are autoreactive to endogenous self-peptides and cause destruction of tissue. Autoimmune diseases include, but are not limited to, acquired factor VIII deficiency, acquired generalized lipodystrophy, alopecia areata, ankylosing spondylitis, anticardiolipin syndrome, autoimmune adrenalitis, autoimmune neutropenia, autoimmune oophoritis, autoimmune orchitis, autoimmune polyendocrine syndrome type 2, autoimmune sclerosing pancreatitis, Balanatis xerotica obliterans, Behcet's disease, benign recurrent meningitis, Calcinosis-Raynaud's sclerodactyl)-telangiectasia syndrome, Caplan's disease, Churg-Strauss syndrome, cicatricial pemphigoid, Degos' disease, dermatitis herpetiformis, discoid lupus erythematosus, Dressler's syndrome, Eaton-Lambert syndrome, eosinophilic fasciitis, eosinophilic pustular folliculitis, epidermolysis bullosa acquisita, Evans syndrome, cryptogenic fibrosing alveolitis, Henoch-Schönlein purpura, Hughes-Stovin syndrome, hypertrophic pulmonary osteo-arthropathy, autoimmune hypoparathyroidism, inclusion body myositis, inflammatory bowel disease, insulin antibodies, insulin receptor antibodies, juvenile chronic arthritis, Kawasaki disease, linear IgA disease, lymphocytic mastisis, microscopic polyangiitis, Mikulicz's syndrome, Miller-Fisher syndrome, morphoea, acquired neuromyotonia, oculovestibuloauditory syndrome, paraneoplastic pemphigus, paroxysmal cold hemoglobinuria, partial lipodystrophy, polyarteritis nodosa, polychondritis, polymyalgia rheumatica, polyradiculoneuropathy, postpartum thyroiditis, primary biliary cirrhosis, primary sclerosing cholangitis, pyoderma gangrenosum, rhizomelic pseudopolyarthritis, sarcoidosis, Sicca syndrome, Sneddon-Wilkinson disease, Still's Disease, Susac's syndrome, sympathetic ophthalmitis, systemic sclerosis, Takayasu's arteritis, temporal arteritis, thrombangiitis obliterans, ulcerative colitis, vitiligo, Vogt-Koyanagi-Harada syndrome, Wegener's granulomatosis, rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Graves' disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjögren's syndrome, insulin resistance, insulin-dependent diabetes mellitus, graft versus host disease, uveitis, rheumatic fever, Guillain-Barre syndrome, psoriasis, and autoimmune hepatitis.

Methods of the invention are particularly useful for COPD, adult (acute) respiratory distress, asthma, cystic fibrosis, emphysema, and bronchopulmonary dysplasia.

Methods for Assaying Modulation of an Immune Response

To evaluate the efficacy of a composition of the invention in modulating an immune response, any standard method known to the skilled artisan may be used. Methods for modulating an immune response are described herein. These include the NFκB Assay described in Example 4, the IκBα assay described in Example 4, and the cytokine release assay described in Example 6. In one embodiment, the methods involve comparing an inflammatory response in a cell or tissue contacted with an RSV Glycoprotein or fragment thereof to the inflammatory response of a corresponding control cell not contacted with the RSV Glycoprotein or fragment. In one embodiment, the inflammatory response is evaluated by comparing the cells gene expression profiles. The gene expression profile of a cell modulated by an RSV Glycoprotein or fragment thereof or analog can be obtained by any of the known in the art or described herein, such methods include but are not limited to microarray analysis, calorimetric assays such as the Bradford Assay and Lowry Assay, RT-PCR, Northern blotting, Western blotting, flow cytometry, immunocytochemistry, binding to magnetic and/or antibody-coated beads, in situ hybridization, fluorescence in situ hybridization (FISH), flow chamber adhesion assay, and ELISA. The protein expression profile of a cell modulated by an RSV Glycoprotein or fragment thereof or analog can be obtained by any of the known in the art or described herein. In particular embodiments, a proteomic protein profile for proteins modulated during an immune response is obtained. In one embodiment, an RSV Glycoprotein reduces the expression of genes upregulated during an adverse immune response. Gene expression modulated in an immune response are known to one skilled in the art. Exemplary genes modulated in an immune response include NFκB, cytokines, IκDα, IL-6, IL1-β, TNFα, CD25, IL-10, IL-8, chemokines, such as RANTES, IL-18, and IL-12.

Changes in tissue or organ morphology as a result of inflammation further comprise values and/or profiles that can be assayed by methods of the invention by any method known in the art, including x-ray, sonogram and ultrasound. In one embodiment, an RSV Glycoprotein ameliorates inflammatory changes associated with an adverse immune response.

Pathogen Infections

Methods of the invention are useful for enhancing a desirable immune response, such as an adaptive immune response against a pathogen. Pathogens include, but are not limited to, bacteria, viruses, fungi, and parasites. Exemplary bacterial pathogens include, but are not limited to, Aerobacter, Aeromonas, Acinetobacter, Actinomyces israelli, Agrobacterium, Bacillus, Bacillus antracis, Bacteroides, Bartonella, Bordetella, Bortella, Borrelia, Brucella, Burkholderia, Calymmatobacterium, Campylobacter, Citrobacter, Clostridium, Clostridium perfringers, Clostridium tetani, Cornyebacterium, Corynebacterium diphtheriae, Corynebacterium sp., Enterobacter, Enterobacter aerogenes, Enterococcus, Erysipelothrix rhusiopathiae, Escherichia, Francisella, Fusobacterium nucleatum, Gardnerella, Haemophilus, Hafnia, Helicobacter, Klebsiella, Klebsiella pneumoniae, Legionella, Leptospira, Listeria, Morganella, Moraxella, Mycobacterium, Neisseria, Pasteurella, Pasturella multocida, Proteus, Providencia, Pseudomonas, Rickettsia, Salmonella, Serratia, Shigella, Staphylococcus, Stentorophomonas, Streptococcus, Streptobacillus monilifommis, Treponema, Treponema pallidium, Treponema pertenue, Xanthomonas, Vibrio, and Yersinia.

Both gram negative and gram positive bacteria may act as pathogens in vertebrate animals. Gram positive bacteria include, but are not limited to, Pasteurella species, Staphylococci species, and Streptococcus species. Gram negative bacteria include, but are not limited to, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israelli.

Examples of viruses that have been found in humans include, but are not limited to, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses, including influenza A, B, and C); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

In particular embodiments, the invention provides compositions that increase an immune response against Orthomyxoviridae, which include influenza viruses, such as influenza A, B, and C.

Examples of pathogenic fungi include, without limitation, Alternaria, Aspergillus, Basidiobolus, Bipolaris, Blastomyces dermatitidis, Blastoschizomyces, Candida, Candida albicans, Candida krusei, Candida glabrata (formerly called Torulopsis glabrata), Candida parapsilosis, Candida tropicalis, Candida pseudotropicalis, Candida guilliermondii, Candida dubliniensis, Candida lusitaniae, Coccidioides, Coccidioides immitis, Cladophialophora, Chlamydia trachomatis, Candida albicans, Cryptococcus, Cryptococcus neoformans, Cunninghamella, Curvularia, Exophiala, Fonsecaea, Histoplasma, Histoplasma capsulatum, Madurella, Malassezia, Plastomyces, Rhodotorula, Scedosporium, Scopulariopsis, Sporobolomyces, Tinea, and Trichosporon.

Examples of parasites include Acanthamoeba, Babesia, Babesia microti, Babesia divergens, Cryptosporidium, Eimeria, Entamoeba histolytica, Enterocytozoon bieneusi Giardia lamblia, Isospora, Leishmania, Leishmania tropica, Leishmania braziliensis, Leishmania donovani, Naegleria, Neospora, Plasmodium, Sarcocystis, and Schistosoma Trypanosoma cruzi, Toxoplasma gondii, and Trichinella spiralis. Exemplary parasitic helminths include nematodes, cestodes, and trematodes. Preferred nematodes include filariid, ascarid, capillarid, strongylid, strongyloides, trichostrongyle, and trichurid nematodes.

Other medically relevant microorganisms have been described extensively in the literature, e.g., see C. G. A Thomas, Medical Microbiology, Bailliere Tindall, Great Britain 1983, the entire contents of which is hereby incorporated by reference.

Accordingly, an embodiment of the invention relates to a method of stabilizing, reducing, or ameliorating a pathogen infection in a subject comprising the steps of:
a) contacting a pathogen cell with a therapeutically effective amount of an RSV Glycoprotein or fragment thereof comprising a GCRR; and
b) stabilizing, reducing, or ameliorating the pathogen infection.

Methods of evaluating a pathogen infection are known in the art and are described in the Examples.

Vaccine Production

The invention also provides for a method of inducing an immunological response in a subject, particularly a human, which comprises inoculating the subject with the polypeptides of the invention, or fragments thereof, in a suitable carrier for the purpose of inducing or enhancing an immune response. In one embodiment, an immune response protects the subject from a pathogen infection, such as a herpes, cytomegalovirus, HIV, AIDs, or a parasite infection. The administration of this immunological composition may be used either therapeutically in subjects already experiencing a pathogen infection, or may be used prophylactically to prevent a pathogen infection. In another embodiment, an immune response treats a neoplasia in a subject in need thereof.

The preparation of vaccines is known to one skilled in the art. The vaccine includes an RSV Glycoprotein or fragment thereof. In one embodiment, the fragment is a GCRR. Alternatively, the vaccine comprises an expression vector encoding an RSV Glycoprotein or fragment thereof or variants thereof. Such a vaccine is delivered in vivo in order to induce or enhance an immunological response comprising a cytotoxic T cell response.

For example, the RSV Glycoprotein, or fragments or variants thereof are delivered in vivo in order to induce an immune response. The polypeptides might be fused to a recombinant protein that stabilizes the polypeptide of the invention, aids in its solubilization, facilitates its production or purification.

Typically vaccines are prepared in an injectable form, either as a liquid solution or as a suspension. Solid forms suitable for injection may also be prepared as emulsions, or with the polypeptides encapsulated in liposomes. Vaccine antigens are usually combined with a pharmaceutically acceptable carrier, which includes any carrier that does not induce the production of antibodies harmful to the subject receiving the carrier. Suitable carriers typically comprise large macromolecules that are slowly metabolized, such as proteins, polysaccharides, polylactic acids, polyglycolic acids, polymeric amino acids, amino acid copolymers, lipid aggregates, and inactive virus particles. Such carriers are well known to those skilled in the art. These carriers may also function as adjuvants.

The RSV Glycoprotein, or fragments or variants thereof are useful as an adjuvant. Adjuvants are immunostimulating agents that enhance vaccine effectiveness. The RSV Glycoprotein, or fragments or variants thereof are administered in combination with an antigen of interest, such that the presence of the RSV Glycoprotein enhances the effectiveness of the immune response generated against the antigen of interest. The RSV Glycoprotein composition may be combined with any other adjuvant known in the art. Effective adjuvants include, but are not limited to, aluminum salts such as aluminum hydroxide and aluminum phosphate, muramyl peptides, bacterial cell wall components, saponin adjuvants, and other substances that act as immunostimulating agents to enhance the effectiveness of the composition.

Immunogenic compositions, i.e. the antigen, pharmaceutically acceptable carrier and adjuvant, also typically contain diluents, such as water, saline, glycerol, ethanol. Auxiliary substances may also be present, such as wetting or emulsifying agents, pH buffering substances, and the like. Proteins may be formulated into the vaccine as neutral or salt forms. The vaccines are typically administered parenterally, by injection; such injection may be either subcutaneously or intramuscularly. Additional formulations are suitable for other forms of administration, such as by suppository or orally. Oral compositions may be administered as a solution, suspension, tablet, pill, capsule, or sustained release formulation.

In addition, it is possible to prepare live attenuated microorganism vaccines that express recombinant polypeptides, for example of an RSV Glycoprotein, fragment thereof, or variant. Suitable attenuated microorganisms are known in the art, and include, for example, viruses and bacteria.

Vaccines are administered in a manner compatible with the dose formulation. The immunogenic composition of the vaccine comprises an immunologically effective amount of the antigenic polypeptides and other previously mentioned components. By an immunologically effective amount is meant a single dose, or a vaccine administered in a multiple dose schedule, that is effective for the treatment or prevention of an infection. The dose administered will vary, depending on the subject to be treated, the subject's health and physical condition, the capacity of the subject's immune system to produce antibodies, the degree of protection desired, and other relevant factors. Precise amounts of the active ingredient required will depend on the judgement of the practitioner, but typically range between 5 μg to 250 μg of antigen per dose.

Polypeptide Expression

In general, polypeptides of the invention may be produced by transformation of a suitable host cell with all or part of a polypeptide-encoding nucleic acid molecule or fragment thereof in a suitable expression vehicle.

Those skilled in the field of molecular biology will understand that any of a wide variety of expression systems may be used to provide the recombinant protein. The precise host cell used is not critical to the invention. A polypeptide of the invention may be produced in a prokaryotic host (e.g., E. coli) or in a eukaryotic host (e.g., Saccharomyces cerevisiae, insect cells, e.g., Sf21 cells, or mammalian cells, e.g., NIH 3T3, HeLa, or preferably COS cells). Such cells are available from a wide range of sources (e.g., the American Type Culture Collection, Rockland, Md.; also, see, e.g., Ausubel et al., supra). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

A variety of expression systems exist for the production of the polypeptides of the invention. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

One particular bacterial expression system for polypeptide production is the E. coli pET expression system (Novagen, Inc., Madison, Wis.). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains that express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system that is designed for high-level expression of genes or gene fragments as fusion proteins with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3× may be cleaved with factor Xa.

Once the recombinant polypeptide of the invention is expressed, it is isolated, e.g., using affinity chromatography. In one example, an antibody (e.g., produced as described herein) raised against a polypeptide of the invention may be attached to a column and used to isolate the recombinant polypeptide. Lysis and fractionation of polypeptide-harboring cells prior to affinity chromatography may be performed by standard methods (see, e.g., Ausubel et al., supra).

Once isolated, the recombinant protein can, if desired, be further purified, e.g., by high performance liquid chromatography (see, e.g., Fisher, Laboratory Techniques In Biochemistry and Molecular Biology, eds., Work and Burdon, Elsevier, 1980). Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.). These general techniques of polypeptide expression and purification can also be used to produce and isolate useful peptide fragments or analogs (described herein).

RSV Glycoproteins and Analogs

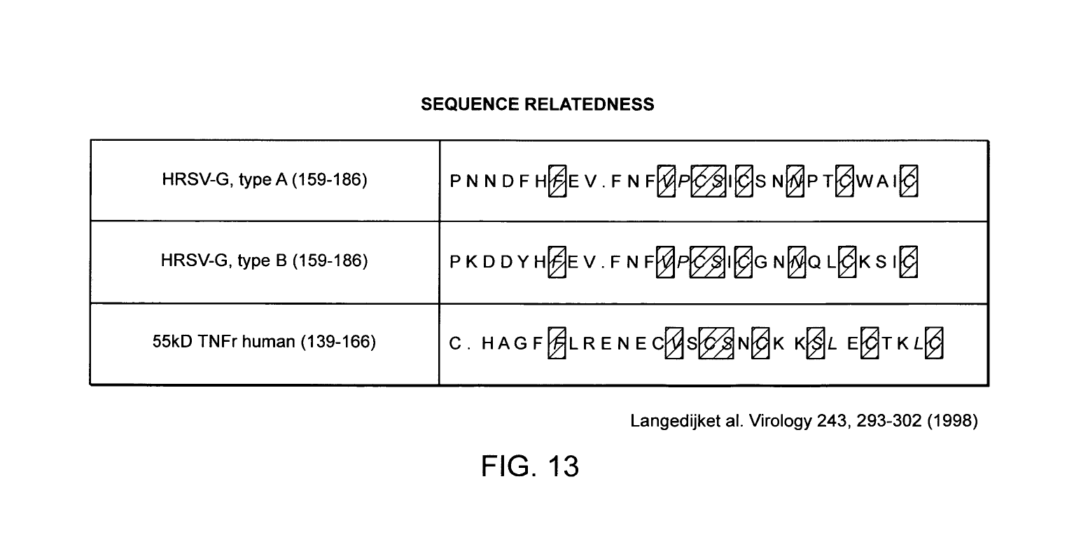

Also included in the invention are RSV Glycoproteins or fragments thereof that are modified in ways that enhance or do not inhibit their ability to modulate an immune response. In one embodiment, the invention provides methods for optimizing an RSV Glycoprotein amino acid sequence or nucleic acid sequence by producing an alteration. FIG. 13 provides an alignment of various Such changes may include certain mutations, deletions, insertions, or post-translational modifications. The invention further includes analogs of any naturally-occurring polypeptide of the invention. Analogs can differ from the naturally-occurring the polypeptide of the invention by amino acid sequence differences, by post-translational modifications, or by both. Analogs of the invention will generally exhibit at least 85%, more preferably 90%, and most preferably 95% or even 99% identity with all or part of a naturally-occurring amino, acid sequence of the invention. The length of sequence comparison is at least 10, 13, 15 amino acid residues, preferably at least 25 amino acid residues, and more preferably more than 35 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the invention by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., .beta. or .gamma. amino acids.

In addition to full-length polypeptides, the invention also includes fragments of any one of the polypeptides of the invention. As used herein, the term “a fragment” means at least 5, 10, 13, or 15. In other embodiments a fragment is at least 20 contiguous amino acids, at least 30 contiguous amino acids, or at least 50 contiguous amino acids, and in other embodiments at least 60 to 80 or more contiguous amino acids. Fragments of the invention can be generated by methods known to those skilled in the art or may result from normal protein processing (e.g., removal of amino acids from the nascent polypeptide that are not required for biological activity or removal of amino acids by alternative mRNA splicing or alternative protein processing events).

Non-protein RSV Glycoprotein analogs having a chemical structure designed to mimic RSV Glycoprotein functional activity can be administered according to methods of the invention. RSV Glycoprotein analogs may exceed the physiological activity of native RSV Glycoproteins. Methods of analog design are well known in the art, and synthesis of analogs can be carried out according to such methods by modifying the chemical structures such that the resultant analogs exhibit the immunomodulatory activity of a native RSV Glycoproeing. These chemical modifications include, but are not limited to, substituting alternative R groups and varying the degree of saturation at specific carbon atoms of the native RSV Glycoprotein molecule. Preferably, the RSV Glycoprotein analogs are relatively resistant to in vivo degradation, resulting in a more prolonged therapeutic effect upon administration. Assays for measuring functional activity include, but are not limited to, those described in the Examples below.

RSV Glycoprotein Polynucleotides

In general, the invention includes any nucleic acid sequence encoding an RSV Glycoprotein fragment comprising at least a GCRR, where the fragment modulates an immune response. An isolated nucleic acid molecule is readily manipulatable by recombinant DNA techniques well known in the art. Thus, a nucleotide sequence contained in a vector in which 5′ and 3′ restriction sites are known, or for which polymerase chain reaction (PCR) primer sequences have been disclosed, is considered isolated, but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, as the term is used herein, because it is readily manipulatable by standard techniques known to those of ordinary skill in the art.

Modulation of an Immune Response using an RSV Glycoprotein Polynucleotide

Polynucleotide therapy featuring a polynucleotide encoding an RSV Glycoprotein or fragment thereof is another therapeutic approach for modulating an immune response or preventing or ameliorating an inflammatory response, an autoimmune response, rejection of a transplanted organ, a neoplasia, or a pathogen infection. Such nucleic acid molecules can be delivered to cells of a subject in need of the modulation of an immune response. The nucleic acid molecules must be delivered to the cells of a subject in a form in which they can be taken up so that therapeutically effective levels of an RSV Glycoprotein or fragment thereof can be produced.

Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding an RSV Glycoprotein or a fragment thereof, can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77 S-83S, 1995). Retroviral vectors are particularly well developed and have been used in clinical settings (Rosenberg et al., N. Engl. J. Med 323:370, 1990; Anderson et al., U.S. Pat. No. 5,399,346). Most preferably, a viral vector is used to administer an RSV Glycoprotein polynucleotide systemically.

Non-viral approaches can also be employed for the introduction of therapeutic to a cell of a patient requiring modulation of an immune response. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal nucleic acid into a cultivatable cell type ex vivo (e.g., an autologous or heterologous primary cell or progeny thereof), after which the cell (or its descendants) are injected into a targeted tissue.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types epithelial cells, dendritic cell, and monocyte macrophages can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Another therapeutic approach included in the invention involves administration of a recombinant therapeutic, such as a recombinant RSV Glycoprotein, or fragment thereof containing a GCRR, either directly to the site of a potential or actual disease-affected tissue or systemically (for example, by any conventional recombinant protein administration technique). The dosage of the administered protein depends on a number of factors, including the size and health of the individual patient. For any particular subject, the specific dosage regimes should be adjusted over time according to the individual need and the professional judgment of the person administering or supervising the administration of the compositions. Generally, between 0.1 mg and 100 mg, is administered per day to an adult in any pharmaceutically acceptable formulation. In particular embodiments, between 0.5 mg and 1 gram may be used. Methods for determining the optimal dosage are within the skill of one in the art.

Screening Assays

As discussed above, an RSV Glycoprotein or fragment thereof is useful for the modulation of an immune response. Accordingly, compounds that enhance the activity of an RSV Glycoprotein or fragment thereof are useful in the methods of the invention. Any number of methods are available for carrying out screening assays to identify such compounds. In one approach, candidate compounds are identified that specifically bind to and enhance the activity of a polypeptide of the invention. In particular, its ability to modulate an immune response. Methods of assaying an immune response are known in the art and are described herein. The efficacy of such a candidate compound is dependent upon its ability to interact with the RSV Glycoprotein. Such an interaction can be readily assayed using any number of standard binding techniques and functional assays (e.g., those described in Ausubel et al., supra). For example, a candidate compound may be tested in vitro for interaction and binding with a polypeptide of the invention and its ability to modulate an immune response may be assayed by any standard assays (e.g., those described herein).

Potential agonists include organic molecules, peptides, peptide mimetics, polypeptides, nucleic acid ligands, and antibodies that bind to a nucleic acid sequence or polypeptide of the invention and thereby inhibit or extinguish its activity. Potential antagonists also include small molecules that bind to and occupy the binding site of the polypeptide thereby preventing binding to cellular binding molecules, such that normal biological activity is prevented.

In one particular example, a candidate compound that binds to RSV Glycoprotein or fragment thereof may be identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for the RSV Glycoprotein is identified on the basis of its ability to bind to the RSV Glycoprotein and be immobilized on the column. To isolate the compound, the column is washed to remove non-specifically bound molecules, and the compound of interest is then released from the column and collected. Compounds isolated by this method (or any other appropriate method) may, if desired, be further purified (e.g., by high performance liquid chromatography). Compounds isolated by this approach may also be used, for example, as therapeutics to treat or prevent the onset of a pathogenic infection, disease, or both. Compounds that are identified as binding to RSV Glycoprotein or fragment thereof with an affinity constant less than or equal to 10 mM are considered particularly useful in the invention.

Optionally, compounds identified in any of the above-described assays may be confirmed as useful in conferring protection against an inflammatory response, a neoplasia, a pathogen infection in any standard animal model and, if successful, may be used as therapeutics.

Test Compounds and Extracts

In general, compounds capable of modulating an immune response or conferring protection against an inflammatory response, a neoplasia, or a pathogen infection by enhancing the activity of an RSV Glycoprotein or fragment thereof are identified from large libraries of either natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their anti-pathogenic activity should be employed whenever possible.

When a crude extract is found to enhance the biological activity of an RSV Glycoprotein, GCRR, or fragment thereof, further fractionation of the positive lead extract is necessary to isolate chemical constituents responsible for the observed effect. Thus, the goal of the extraction, fractionation, and purification process is the careful characterization and identification of a chemical entity within the crude extract having anti-pathogenic activity. Methods of fractionation and purification of such heterogenous extracts are known in the art. If desired, compounds shown to be useful agents for the treatment of pathogenicity are chemically modified according to methods known in the art.

Pharmaceutical Compositions

The present invention contemplates pharmaceutical preparations comprising RSV Glycoprotein molecules or other functional substitutes, such as RSV Glycoprotein analogs, together with pharmaceutically acceptable carriers. Polypeptides of the invention may be administered as part of a pharmaceutical composition. The compositions should be sterile and contain a therapeutically effective amount of the polypeptides in a unit of weight or volume suitable for administration to a subject.

Pharmaceutical compositions of the invention to be used for therapeutic administration should be sterile. Sterility is readily accomplished by filtration through sterile filtration membranes (e.g., 0.2 μm membranes), by gamma irradiation, or any other suitable means known to those skilled in the art. Therapeutic polypeptide compositions generally are placed into a container having a sterile access port, for example, an intravenous solution bag or vial having a stopper pierceable by a hypodermic injection needle. These compositions ordinarily will be stored in unit or multi-dose containers, for example, sealed ampoules or vials, as an aqueous solution or as a lyophilized formulation for reconstitution. As an example of a lyophilized formulation, 10 mL vials are filled with 5 mL of sterile-filtered 1% (w/v) aqueous RSV Glycoprotein solution, such as an aqueous solution of RSV Glycoprotein, and the resulting mixture can then be lyophilized. The infusion solution can be prepared by reconstituting the lyophilized material using sterile Water-for-Injection (WFI).

The polypeptides or analogs may be combined, optionally, with a pharmaceutically acceptable excipient. The term “pharmaceutically-acceptable excipient” as used herein means one or more compatible solid or liquid filler, diluents or encapsulating substances that are suitable for administration into a human. The term “carrier” denotes an organic or inorganic ingredient, natural or synthetic, with which the active ingredient is combined to facilitate administration. The components of the pharmaceutical compositions also are capable of being co-mingled with the molecules of the present invention, and with each other, in a manner such that there is no interaction that would substantially impair the desired pharmaceutical efficacy.

RSV Glycoproteins of the present invention can be contained in a pharmaceutically acceptable excipient. The excipient preferably contains minor amounts of additives such as substances that enhance isotonicity and chemical stability. Such materials are non-toxic to recipients at the dosages and concentrations employed, and include buffers such as phosphate, citrate, succinate, acetate, lactate, tartrate, and other organic acids or their salts; tris-hydroxymethylaminomethane (TRIS), bicarbonate, carbonate, and other organic bases and their salts; antioxidants, such as ascorbic acid; low molecular weight (for example, less than about ten residues) polypeptides, e.g., polyarginine, polylysine, polyglutamate and polyaspartate; proteins, such as serum albumin, gelatin, or immunoglobulins; hydrophilic polymers, such as polyvinylpyrrolidone (PVP), polypropylene glycols (PPGs), and polyethylene glycols (PEGs); amino acids, such as glycine, glutamic acid, aspartic acid, histidine, lysine, or arginine; monosaccharides, disaccharides, and other carbohydrates including cellulose or its derivatives, glucose, mannose, sucrose, dextrins or sulfated carbohydrate derivatives, such as heparin, chondroitin sulfate or dextran sulfate; polyvalent metal ions, such as divalent metal ions including calcium ions, magnesium ions and manganese ions; chelating agents, such as ethylenediamine tetraacetic acid (EDTA); sugar alcohols, such as mannitol or sorbitol; counterions, such as sodium or ammonium; and/or nonionic surfactants, such as polysorbates or poloxamers. Other additives may be included, such as stabilizers, anti-microbials, inert gases, fluid and nutrient replenishers (i.e., Ringer's dextrose), electrolyte replenishers, and the like, which can be present in conventional amounts.

The compositions, as described above, can be administered in effective amounts. The effective amount will depend upon the mode of administration, the particular condition being treated and the desired outcome. It may also depend upon the stage of the condition, the age and physical condition of the subject, the nature of concurrent therapy, if any, and like factors well known to the medical practitioner. For therapeutic applications, it is that amount sufficient to achieve a medically desirable result.

With respect to a subject having an inflammatory disease or disorder, an effective amount is sufficient to reduce an inflammation. In some cases this is a local (site-specific) reduction of inflammation. In other cases, it is inhibition of systemic infection and/or sepsis. With respect to a subject having a neoplastic disease or disorder, an effective amount is an amount sufficient to stabilize, slow, or reduce the proliferation of the neoplasm. Generally, doses of active polypeptide compounds of the present invention would be from about 0.01 mg/kg per day to about 1000 mg/kg per day. It is expected that doses ranging from about 50 to about 2000 mg/kg will be suitable. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of the RSV Glycoprotein compositions of the present invention.

A variety of administration routes are available. The methods of the invention, generally speaking, may be practiced using any mode of administration that is medically acceptable, meaning any mode that produces effective levels of the active compounds without causing clinically unacceptable adverse effects. In one embodiment, a composition of the invention comprising an RSV Glycoprotein or a nucleic acid molecule encoding the RSV Glycoprotein is administered by inhalation. This method of administration is particularly advantageous because it provides the RSV Glycoprotein or nucleic acid molecule directly to the lung epithelium. Other modes of administration include oral, rectal, topical, intraocular, buccal, intravaginal, intracisternal, intracerebroventricular, intratracheal, nasal, transdermal, within/on implants, e.g., fibers such as collagen, osmotic pumps, or grafts comprising appropriately transformed cells, etc., or parenteral routes. A particular method of administration involves coating, embedding or derivatizing fibers, such as collagen fibers, protein polymers, etc. with therapeutic proteins. Other useful approaches are described in Otto, D. et al., J. Neurosci. Res. 22: 83-91 and in Otto, D. and Unsicker, K. J. Neurosci. 10: 1912-1921.

The term “parenteral” includes subcutaneous, intrathecal, intravenous, intramuscular, intraperitoneal, or infusion. Compositions comprising RSV Glycoproteins can be added to a physiological fluid such as blood or synovial fluid. For CNS administration, a variety of techniques are available for promoting transfer of the therapeutic across the blood brain barrier including disruption by surgery or injection, drugs which transiently open adhesion contact between the CNS vasculature endothelial cells, and compounds that facilitate translocation through such cells. Oral administration can be preferred for prophylactic treatment because of the convenience to the patient as well as the dosing schedule.

Pharmaceutical compositions of the invention can optionally further contain one or more additional proteins as desired, including plasma proteins, proteases, and other biological material, so long as it does not cause adverse effects upon administration to a subject. Suitable proteins or biological material may be obtained from human or mammalian plasma by any of the purification methods known and available to those skilled in the art; from supernatants, extracts, or lysates of recombinant tissue culture, viruses, yeast, bacteria, or the like that contain a gene that expresses a human or mammalian plasma protein which has been introduced according to standard recombinant DNA techniques; or from the fluids (e.g., blood, milk, lymph, urine or the like) or transgenic animals that contain a gene that expresses a human plasma protein which has been introduced according to standard transgenic techniques.

Pharmaceutical compositions of the invention can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions of the invention can also contain one or more osmotic modulating agents, i.e., a compound that modulates the osmotic properties (e.g., tonicity, osmolality and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

Compositions comprising RSV Glycoproteins of the present invention can contain multivalent metal ions, such as calcium ions, magnesium ions and/or manganese ions. Any multivalent metal ion that helps stabilize the RSV Glycoprotein composition and that will not adversely affect recipient individuals may be used. The skilled artisan, based on these two criteria, can determine suitable metal ions empirically and suitable sources of such metal ions are known, and include inorganic and organic salts.

Pharmaceutical compositions of the invention can also be a non-aqueous liquid formulation. Any suitable non-aqueous liquid may be employed, provided that it provides stability to the active agents (s) contained therein. Preferably, the non-aqueous liquid is a hydrophilic liquid. Illustrative examples of suitable non-aqueous liquids include: glycerol; dimethyl sulfoxide (DMSO); polydimethylsiloxane (PMS); ethylene glycols, such as ethylene glycol, diethylene glycol, triethylene glycol, polyethylene glycol (“PEG”) 200, PEG 300, and PEG 400; and propylene glycols, such as dipropylene glycol, tripropylene glycol, polypropylene glycol (“PPG”) 425, PPG 725, PPG 1000, PPG 2000, PPG 3000 and PPG 4000.

Pharmaceutical compositions of the invention can also be a mixed aqueous/non-aqueous liquid formulation. Any suitable non-aqueous liquid formulation, such as those described above, can be employed along with any aqueous liquid formulation, such as those described above, provided that the mixed aqueous/non-aqueous liquid formulation provides stability to the RSV Glycoprotein(s) contained therein. Preferably, the non-aqueous liquid in such a formulation is a hydrophilic liquid. Illustrative examples of suitable non-aqueous liquids include: glycerol; DMSO; PMS; ethylene glycols, such as PEG 200, PEG 300, and PEG 400; and propylene glycols, such as PPG 425, PPG 725, PPG 1000, PPG 2000, PPG 3000 and PPG 4000.

Suitable stable formulations can permit storage of the active agents in a frozen or an unfrozen liquid state. Stable liquid formulations can be stored at a temperature of at least −70° C., but can also be stored at higher temperatures of at least 0° C., or between about 0.1° C. and about 42° C., depending on the properties of the composition. It is generally known to the skilled artisan that proteins and polypeptides are sensitive to changes in pH, temperature, and a multiplicity of other factors that may affect therapeutic efficacy.

In certain embodiments a desirable route of administration can be by pulmonary aerosol. Techniques for preparing aerosol delivery systems containing polypeptides are well known to those of skill in the art. Generally, such systems should utilize components that will not significantly impair the biological properties of the antibodies, such as the paratope binding capacity (see, for example, Sciarra and Cutie, “Aerosols,” in Remington's Pharmaceutical Sciences, 18th edition, 1990, pp 1694-1712; incorporated by reference). Those of skill in the art can readily modify the various parameters and conditions for producing polypeptide aerosols without resorting to undue experimentation.

Other delivery systems can include time-release, delayed release or sustained release delivery systems. Such systems can avoid repeated administrations of RSV Glycoproteins, increasing convenience to the subject and the physician. Many types of release delivery systems are available and known to those of ordinary skill in the art. They include polymer base systems such as polylactides (U.S. Pat. No. 3,773,919; European Patent No. 58,481), poly(lactide-glycolide), copolyoxalates, polycaprolactones, polyesteramides, polyorthoesters, polyhydroxybutyric acids, such as poly-D-(-)-3-hydroxybutyric acid (European Patent No. 133, 988), copolymers of L-glutamic acid and gamma-ethyl-L-glutamate (Sidman, K. R. et al., Biopolymers 22: 547-556), poly(2-hydroxyethyl methacrylate) or ethylene vinyl acetate (Langer, R. et al., J. Biomed. Mater. Res. 15:267-277; Langer, R. Chem. Tech. 12:98-105), and polyanhydrides.

Other examples of sustained-release compositions include semi-permeable polymer matrices in the form of shaped articles, e.g., films, or microcapsules. Delivery systems also include non-polymer systems that are: lipids including sterols such as cholesterol, cholesterol esters and fatty acids or neutral fats such as mono- di- and tri-glycerides; hydrogel release systems such as biologically-derived bioresorbable hydrogel (i.e., chitin hydrogels or chitosan hydrogels); sylastic systems; peptide based systems; wax coatings; compressed tablets using conventional binders and excipients; partially fused implants; and the like. Specific examples include, but are not limited to: (a) erosional systems in which the anti-inflammatory agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480.

Another type of delivery system that can be used with the methods and compositions of the invention is a colloidal dispersion system. Colloidal dispersion systems include lipid-based systems including oil-in-water emulsions, micelles, mixed micelles, and liposomes. Liposomes are artificial membrane vessels, which are useful as a delivery vector in vivo or in vitro. Large unilamellar vessels (LUV), which range in size from 0.2-4.0 μm, can encapsulate large macromolecules within the aqueous interior and be delivered to cells in a biologically active form (Fraley, R., and Papahadjopoulos, D., Trends Biochem. Sci. 6: 77-80).

Liposomes can be targeted to a particular tissue by coupling the liposome to a specific ligand such as a monoclonal antibody, sugar, glycolipid, or protein. Liposomes are commercially available from Gibco BRL, for example, as LIPOFECTIN™ and LIPOFECTACE™, which are formed of cationic lipids such as N-[1-(2,3 dioleyloxy)-propyl]-N,N, N-trimethylammonium chloride (DOTMA) and dimethyl dioctadecylammonium bromide (DDAB). Methods for making liposomes are well known in the art and have been described in many publications, for example, in DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Liposomes also have been reviewed by Gregoriadis, G., Trends Biotechnol., 3: 235-241).

Another type of vehicle is a biocompatible microparticle or implant that is suitable for implantation into the mammalian recipient. Exemplary bioerodible implants that are useful in accordance with this method are described in PCT International application no. PCT/US/03307 (Publication No. WO 95/24929, entitled “Polymeric Gene Delivery System”). PCT/US/0307 describes biocompatible, preferably biodegradable polymeric matrices for containing an exogenous gene under the control of an appropriate promoter. The polymeric matrices can be used to achieve sustained release of the exogenous gene or gene product in the subject.

The polymeric matrix preferably is in the form of a microparticle such as a microsphere (wherein an agent is dispersed throughout a solid polymeric matrix) or a microcapsule (wherein an agent is stored in the core of a polymeric shell). Microcapsules of the foregoing polymers containing drugs are described in, for example, U.S. Pat. No. 5,075,109. Other forms of the polymeric matrix for containing an agent include films, coatings, gels, implants, and stents. The size and composition of the polymeric matrix device is selected to result in favorable release kinetics in the tissue into which the matrix is introduced. The size of the polymeric matrix further is selected according to the method of delivery that is to be used. Preferably, when an aerosol route is used the polymeric matrix and RSV Glycoproteins are encompassed in a surfactant vehicle. The polymeric matrix composition can be selected to have both favorable degradation rates and also to be formed of a material, which is a bioadhesive, to further increase the effectiveness of transfer. The matrix composition also can be selected not to degrade, but rather to release by diffusion over an extended period of time. The delivery system can also be a biocompatible microsphere that is suitable for local, site-specific delivery. Such microspheres are disclosed in Chickering, D. E., et al., Biotechnol. Bioeng., 52: 96-101; Mathiowitz, E., et al., Nature 386: 410-414.

Both non-biodegradable and biodegradable polymeric matrices can be used to deliver the RSV Glycoprotein compositions of the invention to the subject. Such polymers may be natural or synthetic polymers. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene, polyvinylpyrrolidone, and polymers of lactic acid and glycolic acid, polyanhydrides, poly(ortho)esters, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Combination Therapies for the Treatment of Inflammation

Compositions and methods of the invention can be used in combination with existing anti-inflammatory treatment modalities, including but not limited to, drug therapy, and administration with anti-inflammatory cytokines. Methods of the invention can optionally comprise contacting inflammatory cells with RSV Glycoproteins in combination with other anti-inflammatory drug treatments such as, but not limited to, antihistamines, non-steroidal anti-inflammatory agents (NSAIDs), eicosanoid receptor antagonists, cytokine antagonists, monoclonal antibodies, 3-hydroxymethylglutaryl-coenzyme A (HMG-CoA) reductase inhibitors, and corticosteroids (see, for example, Goodman and Gilman's The Pharmacological Basis of Therapeutics).

Antihistamines fall generally under three broad classes, according to the histamine receptor subtype they antagonize and display specificity for. Histamine H1 receptors are primarily responsible for the anti-inflammatory response, while H2 receptors are limited to gastric acid secretion. Histamine H1 receptor antagonists include, but are not limited to, carbinoxamine, clemastine, diphenhydramine, dimenhydrinate, pyrilamine, tripelennamine, chlorpheniramine, brompheniramine, chlorcyclizine, acrivastine, promethazine, as well as piperazines such as astemizole, levocabastine, hydroxyzine, cyclizine, cetirizine, meclizine, loratadine, fexofenadine, and terfenadine.

NSAIDs include the salicylate derivatives, para-aminophenol derivatives, indole and indene acetic acids, heteroaryl acetic acids, arylpropionic acids, anthranilic acids (also known in the art as fenamates), enolic acids, and alkanones. Salicylate derivates include aspirin, sodium salicylate, choline magnesium trisalicylate, salsalate, diflunisal, salicylsalicylic acid, sulfasalazine, and olsalazine, but are not limited to these drugs. Para-aminophenol derivates are exemplified by acetaminophen. Indomethacin, sulindac, and etodolac comprise indole and indene acetic acids, while heteroaryl acetic acids include tolmetin, diclofenac, and ketorolac. Examples of arylpropionic acids include ibuprofen, naproxen, flurbiprofen, ketoprofen, fenoprofen, and oxaprozin. Fenamates include but are not limited to mefenamic acid and meclofenamic acid. Some examples of enolic acids include the oxicams piroxicm and tenoxicam, and pyrazolidinediones such as phenylbutazone and oxyphenthatrazone. Alkanones can comprise nabumetone.

Eicosanoid receptor antagonists include, but are not limited to, leukotriene modifiers, which can act as leukotriene receptor antagonists by selectively competing for LTD-4 and LTE-4 receptors. These compounds include, but are not limited to, zafirlukast tablets, zileuton tablets, and montelukast. Zileuton tablets function as 5-lipoxygenase inhibitors. Cytokine antagonists can comprise anti-TNFα antibodies, and fusion proteins of the ligand binding domain of the TNFα receptor and the Fc portion of human immunoglobulin G1. Other cytokine antagonists include recombinant human interleukin-1 receptor antagonist, recombinant human IFNα, recombinant human IFNβ, IL-4 muteins, soluble IL-4 receptors, immunosuppressants (such as tolerizing peptide vaccine), anti-IL-4 antibodies, IL-4 antagonists, anti-IL-5 antibodies, soluble IL-13 receptor-Fc fusion proteins, anti-IL-9 antibodies, CCR3 antagonists, CCR5 antagonists, VLA-4 inhibitors, downregulators of IgE, among others.

Corticosteroids cause a decrease in the number of circulating lymphocytes as a result of steroid-induced lysis of lymphocytes, or by alterations in lymphocyte circulation patterns (Kuby, J. (1998) In: Immunology 3^{rd} Edition W.H. Freeman and Company, New York; Pelaia, G. et al. Life Sci. 72(14): 1549-61). Corticosteroids affect the regulation of nuclear factor κB (NF-κB) by inducing the upregulation of an inhibitor of NF-κB known as IκB, which sequesters NF-κB in the cytoplasm and prevents it from transactivating pro-inflammatory genes in the nucleus. Corticosteroids also reduce the phagocytic ability of macrophages and neutrophils, as well as reducing chemotaxis. Examples of corticosteroids are alclometasone, amcinonide, beclomethasone, betamethasone, clobetasol, clocortolone, cortisol, hydrocortisone, prednisolone, and prednisone, but are not limited to these examples.

Methods of the invention can optionally comprise contacting inflammatory cells with RSV Glycoproteins in combination with other anti-inflammatory cytokines such as, but not limited to, interleukin-4 (IL-4), interleukin-10 (IL-10), interleukin-13 (IL-13), interleukin-16 (IL-16), interleukin-1 receptor antagonist (IL-1ra), interferon α (IFNα), transforming growth factor-β(TGF-β, among others. The cytokines may be administered together or separately in combination with RSV Glycoproteins in the compositions and methods described herein.

The balance between pro-inflammatory cytokines and anti-inflammatory cytokines determines the net effect of an inflammatory response. The type, duration, and also the extent of cellular activities induced by one particular cytokine can be influenced considerably by the nature of the target cells, the micro-environment of a cell, depending, for example, on the growth and activation state of the cells, the type of neighboring cells, cytokine concentrations, the presence of other cytokines, and even on the temporal sequence of several cytokines acting on the same cell.

Combination Therapy for the Treatment of a Neoplasm

Compositions and methods of the invention may be used in combination with any conventional therapy known in the art. In one embodiment, an RSV Glycoprotein composition of the invention that targets a neoplastic cell may be used in combination with any anti-neoplastic therapy known in the art. Exemplary anti-neoplastic therapies include, for example, chemotherapy, cryotherapy, hormone therapy, radiotherapy, and surgery. A RSV Glycoprotein composition of the invention may, if desired, include one or more chemotherapeutics typically used in the treatment of a neoplasm, such as abiraterone acetate, altretamine, anhydrovinblastine, auristatin, bexarotene, bicalutamide, BMS184476, 2,3,4,5,6-pentafluoro-N-(3-fluoro-4-methoxyphenyl)benzene sulfonamide, bleomycin, N,N-dimethyl-L-valyl-L-valyl-N-methyl-L-valyl-L-proly-1-Lproline-t-butylamide, cachectin, cemadotin, chlorambucil, cyclophosphamide, 3′,4′-didehydro-4′-deoxy-8′-norvin-caleukoblastine, docetaxol, doxetaxel, cyclophosphamide, carboplatin, carmustine (BCNU), cisplatin, cryptophycin, cyclophosphamide, cytarabine, dacarbazine (DTIC), dactinomycin, daunorubicin, dolastatin, doxorubicin (adriamycin), etoposide, 5-fluorouracil, finasteride, flutamide, hydroxyurea and hydroxyureataxanes, ifosfamide, liarozole, lonidamine, lomustine (CCNU), mechlorethamine (nitrogen mustard), melphalan, mivobulin isethionate, rhizoxin, sertenef, streptozocin, mitomycin, methotrexate, 5-fluorouracil, nilutamide, onapristone, paclitaxel, prednimustine, procarbazine, RPR109881, stramustine phosphate, tamoxifen, tasonermin, taxol, tretinoin, vinblastine, vincristine, vindesine sulfate, and vinflunine. Other examples of chemotherapeutic agents can be found in Cancer Principles and Practice of Oncology by V. T. Devita and S. Hellman (editors), 6.sup.th edition (Feb. 15, 2001), Lippincott Williams & Wilkins Publishers.

Combination Therapy for the Treatment of a Pathogen Infection

In another embodiment, a RSV Glycoprotein composition of the invention that targets a pathogen cell may be used in combination with any anti-pathogen therapy known in the art. Exemplary anti-pathogen therapies include antibiotics, antivirals, fungicides, nematicides, and parasiticides, or any other biocide. Parasiticides are agents that kill parasites directly and can be used in combination with the methods and compositions described herein. Such compounds are known in the art and are generally commercially available. Exemplary parasiticides useful for human administration include but are not limited to albendazole, amphotericin B, benznidazole, bithionol, chloroquine HCl, chloroquine phosphate, clindamycin, dehydroemetine, diethylcarbamazine, diloxanide furoate, eflomithine, furazolidaone, glucocorticoids, halofantrine, iodoquinol, ivermectin, mebendazole, mefloquine, meglumine antimoniate, melarsoprol, metrifonate, metronidazole, niclosamide, nifurtimox, oxamniquine, paromomycin, pentamidine isethionate, piperazine, praziquantel, primaquine phosphate, proguanil, pyrantel pamoate, pyrimethanmine-sulfonamides, pyrimethanmine-sulfadoxine, quinacrine HCl, quinine sulfate, quinidine gluconate, spiramycin, stibogluconate sodium (sodium antimony gluconate), suramin, tetracycline, doxycycline, thiabendazole, tinidazole, trimethroprim-sulfamethoxazole, and tryparsamide some of which are used alone or in combination with others.

Other anti-pathogen therapeutics useful in combination with a method of the invention include, but are not limited to, any one or more of the following: agent which reduces the activity of or kills a microorganism and includes but is not limited to Aztreonam; Chlorhexidine Gluconate; Imidurea; Lycetamine; Nibroxane; Pirazmonam Sodium; Propionic Acid; Pyrithione Sodium; Sanguinarium Chloride; Tigemonam Dicholine; Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefinenoxime Hydrochloride; Cefmetazole; Cefmetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride, Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin lydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacil; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz: Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium: Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; Zorbamycin; Difloxacin Hydrochloride; Lauryl Isoquinolinium Bromide; Moxalactam Disodium; Omidazole; Pentisomicin; and Sarafloxacin Hydrochloride.

RSV Viral Fusion Glycoprotein

An important mechanism known to determine severity of disease during RSV infection is the immune response, of which innate immunity is an important component^{1,2} While host immunity clearly is important for restricting and resolving RSV infection, it also is thought to contribute to RSV disease. The RSV viral fusion (F) glycoprotein interacts with the CD 14⁺/Toll-like receptor 4 (TLR4) complex in monocytes and stimulates production of proinflammatory cytokines, such as interleukin-6 (IL-6), IL1β and IL-8 (Kurt-Jones et al., Nat. Immunol. 1:398-401, 2000), by promoting nuclear translocation of NF-κB⁴. These pro inflammatory cytokines play an important role in neutrophil and macrophage chemotaxis and activation during RSV disease. Furthermore, the cellular inflammatory response during severe lung disease in RSV-infected infants is composed overwhelmingly of neutrophils and macrophages⁵, and loss-of-function mutations or polymorphisms in TLR4 affect severity of disease in mice and is associated with severe disease in humans^{2,6}. The interaction of RSV with CD14⁺/TLR4 also promotes increased pulmonary infiltration with natural killer (NK) cells and is important for viral clearance after infection².

In addition to stimulating innate immunity, F elicits neutralizing antibody against RSV 3, has cytotoxic T lymphocyte (CTL) epitopes in mice and humans^{7,8} and has been associated with increased production of Th1 cytokines⁹. Conversely, the other neutralization antigen, RSV attachment protein (Glycoprotein)⁴, is not a strong agonist of the CD14⁺/TLR4 complex³, does not stimulate CTL activity^{7,8} and primes for a Th2 response upon RSV infection^{9,10}. No role is currently recognized for the Glycoprotein in innate immunity. Interestingly, addition of Glycoprotein to F protein during immunization in mice decreases production of interferon-γ by up to 70-fold upon RSV challenge when compared to immunization with F alone⁹. In addition, infection of BALB/c mice with an RSV mutant lacking the Glycoprotein and SH genes increases NK and neutrophil trafficking to the lungs compared to control mice infected with a strain of RSV that has Glycoprotein and SH¹¹. These findings may suggest that RSV Glycoprotein modulates innate inflammation.

RSV Glycoprotein

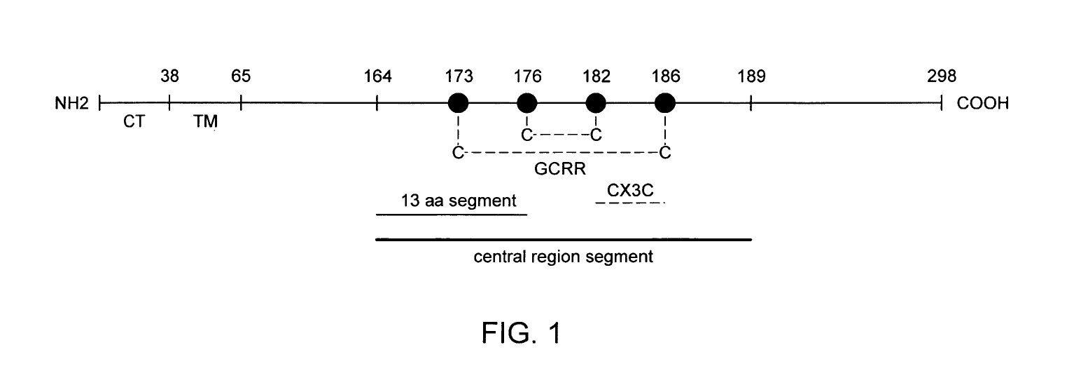

The RSV Glycoprotein is produced as a transmembrane form with an N-terminal cytoplasmatic tail and an N-terminally proximal hydrophobic signal anchor, and as an N-terminally truncated soluble form that is rapidly secreted^{3,12}. Although the secreted form accounts for no more than 20% of the total Glycoprotein synthesized in cell culture through the course of infection, secreted Glycoprotein represents approximately 80% of the protein released into the medium early in infection, during the first twenty-four hours¹². The secreted form was hypothesized to serve as a decoy to saturate the anti-RSV antibody response³, but the timing of its release also suggests that it might be targeted to modulate a very early event, like TLR4-mediated innate immunity. The ectodomain of the Glycoprotein consists of two mucin-like domains, with divergent amino acid sequences between isolates, separated by a short, circumscribed central region that is highly conserved between RSV antigenic subgroups A and B¹². This conserved region includes a 13 amino acid segment (aa 164-176) that is identical in all wild type isolates of RSV and overlaps four cysteine residues (positions 173,176,182 and 186) held by disulfide bonds between 173-186 and 176-182 (FIG. 1). The conservation of the 13 amino acid segment and the cystine rich region (GCRR) among RSV isolates originally had been interpreted as indicating a role in receptor binding, but recent data have shown that they are not required for efficient infection in vitro and in mice^{14,15}. As a consequence, the reason for early secretion of RSV Glycoprotein and the role of its conserved GCRR remain unexplained.

### EXAMPLES

As reported in more detail below, RSV Glycoprotein, through its GCRR, antagonizes the pro-inflammatory effect of RSV F regulating the innate immune response. Furthermore, the Glycoprotein has a similar effect on the unrelated TLR4 agonist TLS, indicating that the GCRR has broad anti-inflammatory properties.

### Example 1

### The Central Region of RSV Glycoprotein Inhibits Production of Inflammatory Cytokines In Vitro

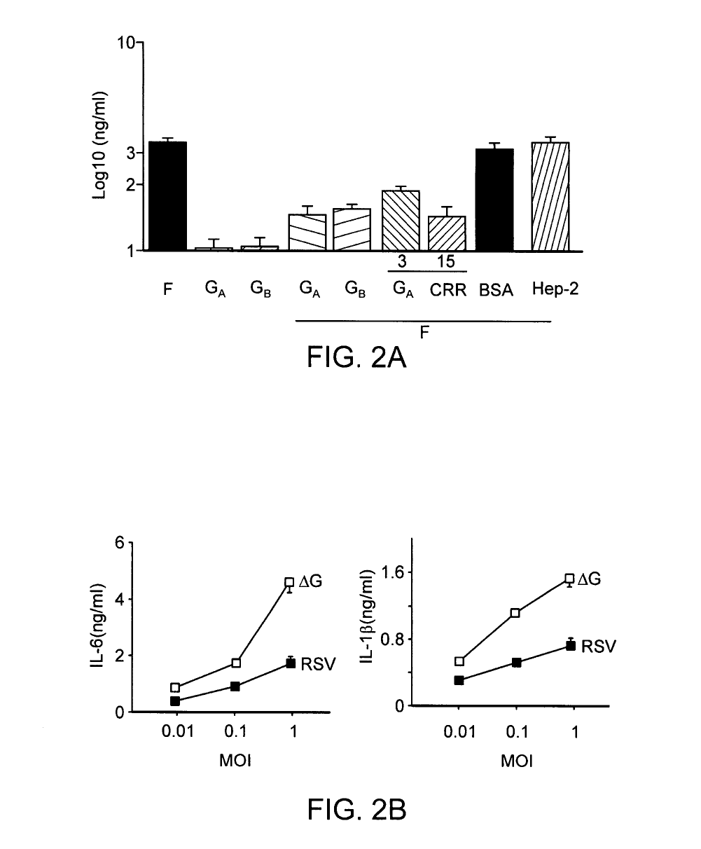

To determine whether the RSV Glycoprotein can inhibit F-mediated monocyte production of pro inflammatory cytokines, purified human monocytes were incubated with purified protein F, purified Glycoprotein from subgroup A or a combination of F+Glycoprotein_{A} and examined supernatant fluids for production of IL-6 (FIG. 2A). Incubation of monocytes with F elicited high levels of IL-6, while levels were low after incubation with Glycoprotein. Monocytes incubated with both proteins decreased IL-6 production by approximately 1.5 log compared to monocytes incubated with F alone (P<0.01). In contrast, addition of equivalent amounts of bovine serum albumin or Hep-2 cell lysate to F-treated monocytes had no effect on IL-6 production. An inhibitory effect similar to that observed for Glycoprotein on F-induced IL-6 production was also detected when IL-1β or IL-10 were measured in supernatant fluids (not shown), and when the Glycoprotein from RSV antigenic subgroup B was used instead of the Glycoprotein from the RSV A2 strain of subgroup A (FIG. 2A).

Inhibition of inflammatory cytokine production elicited by Glycoproteins A and B despite the overall divergence in amino acid sequence between the two proteins^{4,13} suggested that the domain exerting the modulatory effect could localize to the conserved central segment of both proteins (FIG. 1). To determine whether the modulatory effect on cytokine production was elicited by this conserved central region, purified human monocytes were incubated with F protein in combination with increasing concentrations of a synthetic peptide representing amino acids 164-189 of the RSV Glycoprotein_{A}, which includes the conserved 13 amino acid segment and the GCRR (FIG. 2A). Interestingly, increasing concentrations of this peptide led to a dose-dependent inhibition of F-mediated IL-6 production. In contrast, the inhibitory effect was not observed when another Glycoprotein_{A} peptide (residues 273-288) within the Glycoprotein mucin-like domain or an unrelated V3 loop peptide from human immunodeficiency virus type 1 (residues 307-321) were added with F as controls (not shown).

### Example 2

### The Glycoprotein Inhibits Monocyte Production of Inflammatory Cytokines Upon Exposure to RSV

To determine whether the RSV Glycoprotein also inhibited cytokine production during RSV infection, purified monocytes were incubated with increasing concentrations of wild-type RSV or with a live recombinant (r) RSV that does not express the Glycoprotein (ΔG)¹⁶ and supernatant fluids were assayed for inflammatory cytokines eighteen hours later (FIG. 2B). Exposure of monocytes to live ΔG increased production of IL-6 and IL-β in a dose-dependent manner compared to exposure to an identical RSV with normal intact Glycoprotein (FIG. 2B). Differences between viruses were highest at an MOI of 1, which was therefore used for subsequent experiments.

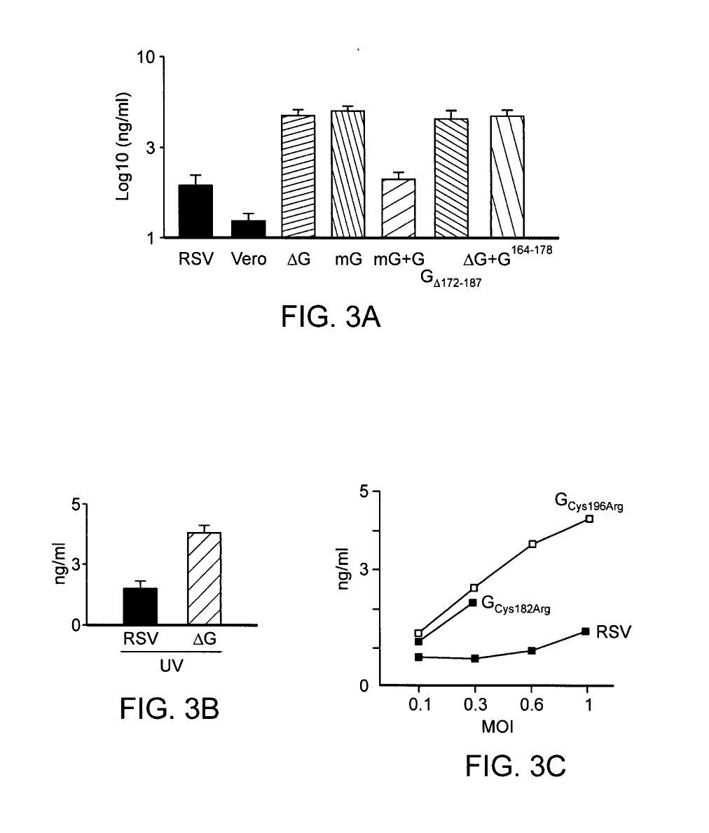

To determine whether the secreted form of the Glycoprotein was responsible for the modulatory effect observed during live RSV infection, purified monocytes were incubated with a recombinant RSV (rRSV) in which the Glycoprotein gene was modified to express only the membrane-anchored (mG) form¹⁶ (FIG. 3A). Incubation of monocytes with mG enhanced IL-6 production to similar levels compared to ΔG, demonstrating that the secreted form of Glycoprotein is required to modulate production of inflammatory cytokines during live RSV infection. Importantly, the IL-6 response was restored to levels similar to those elicited by RSV when soluble Glycoprotein was added back to the culture media of mG-infected cells (FIG. 3A).

Subsequently, to determine whether live RSV infection is required for the observed modulatory effects, human monocytes were incubated with UV-inactivated RSV and ΔG and measured cytokine production (FIG. 3B). Again, ΔG led to enhanced IL-6 production compared to RSV demonstrating that live infection is not necessary for Glycoprotein modulation of the monocyte inflammatory response.

### Example 3

### The Conserved GCRR is Critical for the Inhibitory Effect

The 13 amino acid segment between positions 164-176 (G⁶⁴⁻¹⁷⁶) is a conserved segment in the ectodomain of the Glycoprotein, and it overlaps a GCRR located between positions 173-186. The cysteine residues in this sequence are invariant in both RSV subgroups A and B⁴. To further elucidate the inhibitory role of the conserved central segment of the Glycoprotein, purified human, monocytes were incubated with a rRSV that lacked the GCRR (G_{Δ172-187}) (FIG. 3A). Incubation with G_{Δ172-187} led to higher levels of IL-6 than incubation with RSV, demonstrating that the GCRR modulates innate inflammatory responses in this model. Similar results were obtained with UV-inactivated viruses (not shown). The importance of the cysteine residues on the modulatory effect exerted by Glycoprotein was further examined by incubating human monocytes with two RSV mutants containing substitutions at cysteine residues 182 (G_{Cys182Arg}) or 186 (_{Cys186Arg}) (FIG. 3C). Each of these viruses, G_{Cys182Arg} and G_{Cys186Arg}, present substitutions disrupting one of the two disulfide bridges in the GCRR (FIG. 1). Interestingly, both G_{Cys182Arg} and G_{Cys186Arg} elicited higher IL-6 levels that their RSV control (Rueda, et al., Virology 198:653-662, 1994) at every MOI tested, confirming that the presence of the cysteine residues is essential for the modulatory effect displayed by the GCRR.

To investigate whether the fractalkine motif in the GCRR (FIG. 1) played a role in the modulatory effect on cytokine production, human monocytes were incubated with RSV and a polyclonal anti-CX3CR1 antibody known to block binding of the fractalkine motif in the Glycoprotein to the CX3CR1 chemokine receptor¹⁸. Production of IL-6 was identical in presence or absence of anti-CX3CR1 antibody. Finally, to explore the role of the conserved 13 amino acid segment upstream of the GCRR in modulation of inflammatory cytokines, monocytes were incubated with ΔG in presence of a Glycoprotein peptide encompassing amino acids 164-176 (G¹⁶⁴⁻¹⁷⁶) (FIG. 3A). G¹⁶⁴⁻¹⁷⁶ failed to modulate IL-6 production by ΔG, indicating that the 13 amino acid peptide upstream of the GCRR provided in trans cannot complement the inhibitory effect of the Glycoprotein during live infection. Similar results were observed when using G¹⁶⁴⁻¹⁷⁶ with mG (not shown).

### Example 4

### The Glycoprotein Decreases Nuclear Translocation of the NF-κB Transcription Factor

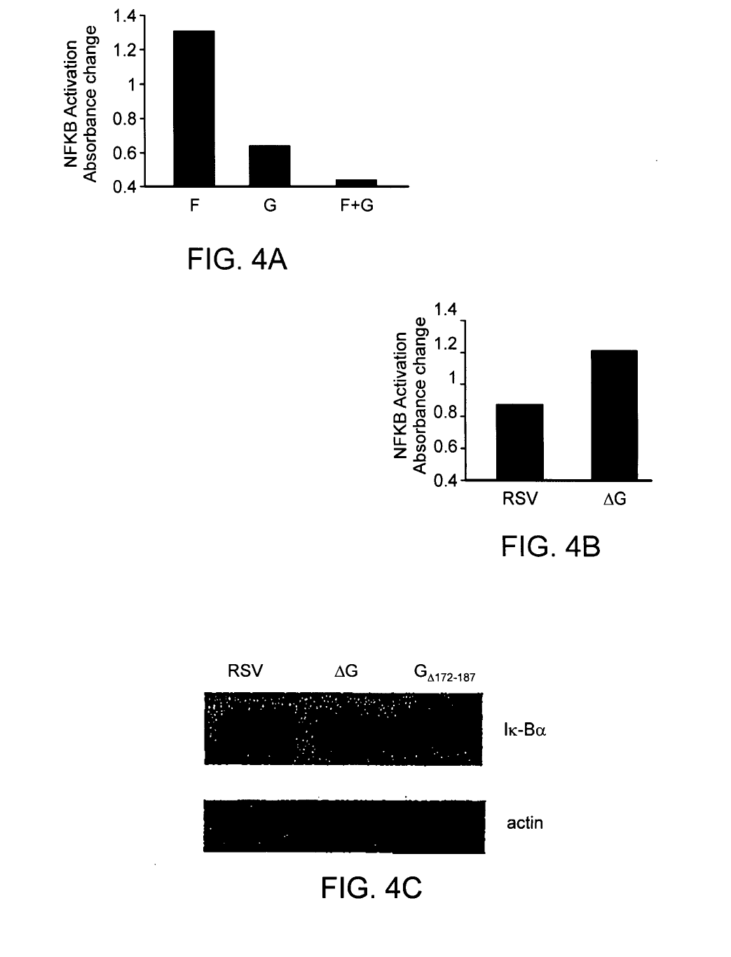

To determine whether the inhibitory effect of the Glycoprotein on inflammatory cytokine production is associated with decreased nuclear translocation of NF-κB, human monocytes were incubated with F protein, Glycoprotein, F+Glycoprotein, and with either RSV or ΔG. The nuclei were then extracted to measure translocation of NF-κB (FIGS. 4A and 4B). NF-κB nuclear translocation was decreased in the presence of the Glycoprotein either after purified protein or live virus stimulation. In addition, IκBα blots from extracts of human monocytes after stimulation with RSV, ΔG or G_{Δ172-187} showed that degradation of IκBα was greater in cells stimulated with ΔG or G_{Δ172-187} than in those incubated with RSV (FIG. 4C). Taken together, these results demonstrate that RSV Glycoprotein, through its GCRR, inhibits nuclear translocation of NF-κB to modulate the innate inflammatory response during RSV infection.

### Example 5

### RSV Glycoprotein Decreases Inflammation During RSV Infection In Vivo

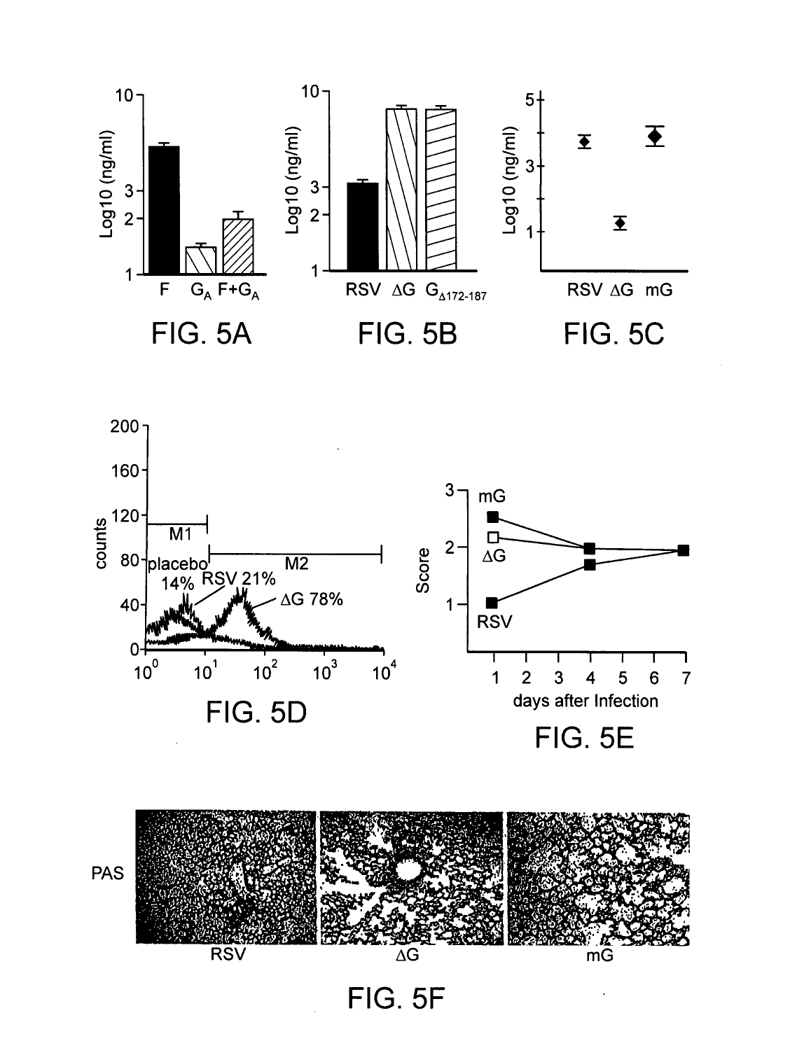

To determine whether the modulatory effect of RSV Glycoprotein affected the innate immune response in vivo, alveolar macrophages were obtained from naive mice and incubated with purified F, Glycoprotein and recombinant viruses (FIGS. 5A and 5B). Cytokine production in murine alveolar macrophages mimicked the response previously observed in human monocytes (FIG. 2), with the Glycoprotein modulating the innate responses elicited by both purified F and RSV. We subsequently infected mice intranasally with RSV or ΔG and measured intracellular production of IL-6 in pulmonary macrophages after infection. ΔG increased production of intracellular IL-6 in pulmonary macrophages compared to RSV (79% vs. 21% of purified macrophages in FIG. 5A) twenty-four hours after infection.

To determine whether this increase in production of inflammatory cytokines after ΔG infection was associated with increased replication of ΔG in the lungs of mice compared to RSV, lung titers of both viruses in infected animals were measured (FIGS. 5C and 5D). Consistent with previous findings^{15,16}, RSV replicated to higher titers in lungs of mice than ΔG (P=0.001). Thus, the proinflammatory effect of ΔG is not associated with heightened replication in the respiratory tract, but rather occurred despite a dramatic decrease in replication. Conversely, replication of mG was similar to RSV (P>0.05) (FIG. 5C) despite eliciting different inflammatory responses (see below, FIGS. 5E and 5F).

To further examine the effect of the Glycoprotein in vivo on the innate immune response, lung sections of mice infected with RSV, ΔG and mG were stained with periodic acid Schiff (PAS) to compare the degree of pulmonary granulocyte and mononuclear cell infiltration on days 1, 4 and 7 after infection (FIGS. 5E and 5F). Considering that replication levels may affect histopathology¹⁹, mG in addition to ΔG was selected because unlike the highly restricted replication of ΔG, replication of mG is not restricted in lungs of mice¹⁶. Furthermore, the majority of Glycoprotein early after infection is secreted and therefore absent in mG^{13,16}. As shown in FIGS. 5E and 5F, early after infection granulocyte and macrophage alveolar infiltration was increased in mice infected with mG and ΔG compared to mice infected with RSV. Mice infected with ΔG had focal areas of increased alveolar inflammation, while the neutrophil and macrophage infiltration in mG recipients was more diffuse. The inflammatory response elicited by the three viruses leveled seven days after infection, when adaptive responses are an important component of the immune response.

### Example 6

### The GCRR Inhibits Endotoxin-Mediated Cytokine Production

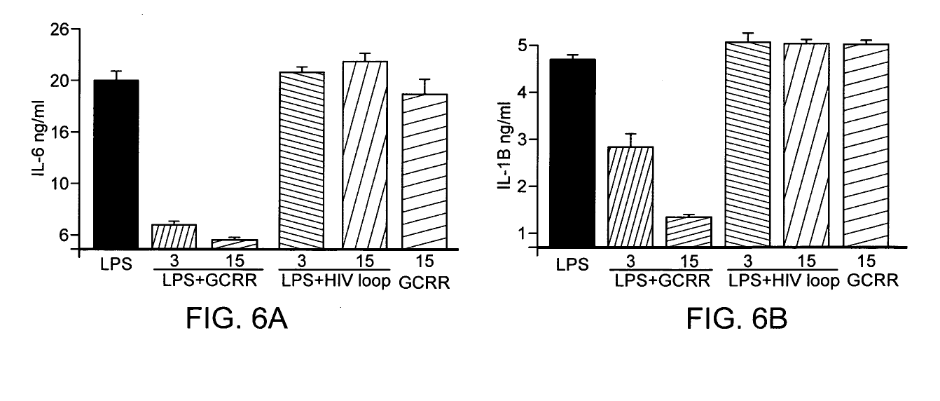

The ability of the GCRR to antagonize the production of pro-inflammatory cytokines in RSV-infected or F protein-exposed monocytes suggested that this protein region could inhibit inflammatory responses elicited by other CD14⁺/TLR4 agonists. Therefore, to examine whether addition of the GCRR peptide inhibited production of cytokines in monocytes stimulated by LPS, purified human monocytes were incubated with LPS and increasing concentrations of the G_{A} central region peptide (aa 164-189) and cytokine production was measured (FIGS. 6A and 6B). As expected, incubation of monocytes with LPS alone elicited high levels of IL-6 and IL-1-β in supernatant fluid. Remarkably, addition of the GCRR peptide to the LPS-treated monocytes caused a dose-dependent inhibition of cytokine production. Addition of an unrelated HIV V3 loop control peptide or a modified central region peptide in which the four cysteine residues had been replaced by serines (GSRR), and therefore lacked disulfide bridges, had no effect (FIGS. 6A and 6B). Similar results were observed when incubating LPS with a scrambled peptide containing the same amino acid composition as GCRR, but in random order (VFNHFECSIFVPCSNRICWANPTICK). Addition of the GCRR peptide to monocytes incubated with LPS also affected production of IL-10. These findings demonstrate that the GCRR not only modulates RSV-mediated inflammation, but also antagonizes LPS-mediated production of inflammatory cytokines.

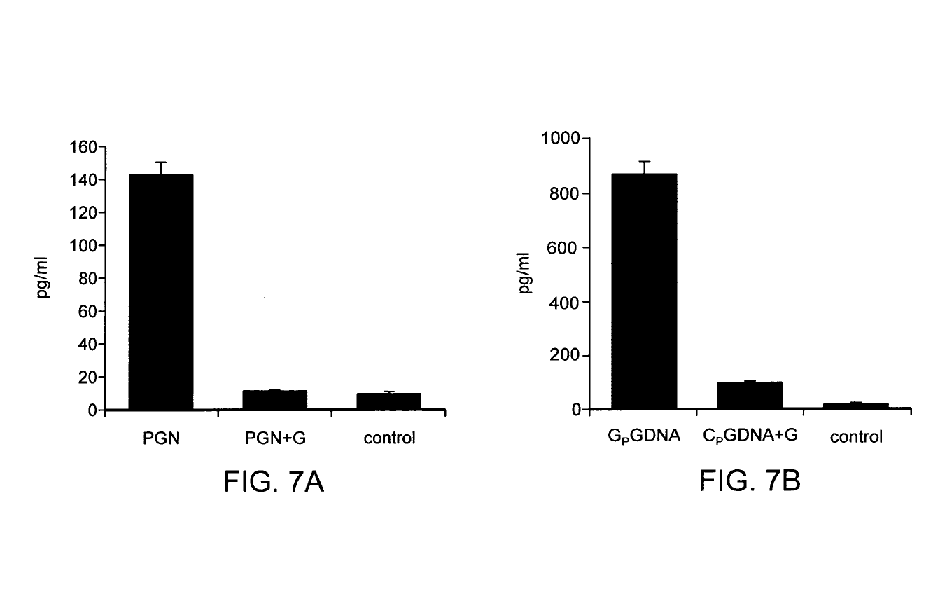

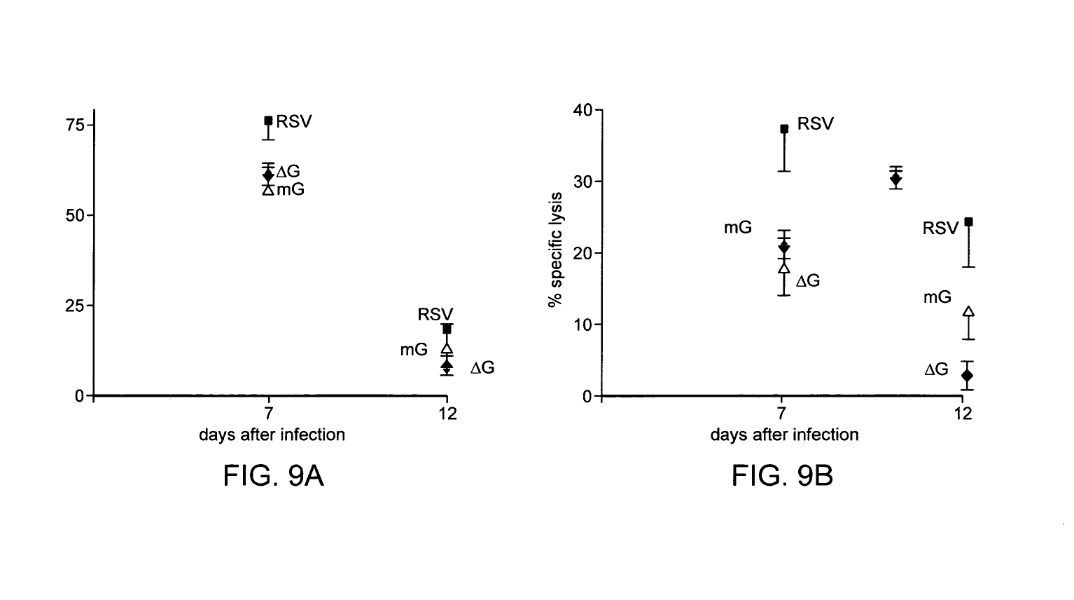

Finally, to examine whether the Glycoprotein affected the inflammatory response elicited by other molecules involved in innate inflammation, human monocytes were incubated with the TLR2 agonist PGN and the TLR9 agonist CpGDNA and cytokine production was examined with or without co incubation with the Glycoprotein (FIG. 9). As previously observed with LPS, cytokine production was inhibited by the Glycoprotein, showing that this molecule is able to counteract a variety of pro inflammatory stimuli. Modulation of TLR2- and TLR9-mediated inflammatory responses is also shown in FIG. 7.

These experiments have identified a novel role for the conserved GCRR of RSV, a region that is highly conserved in wild-type isolates^{4,13,14}. As reported herein, the RSV Glycoprotein modulates monocyte/macrophage cytokine production by inhibiting nuclear translocation of NF-κB, and decreases inflammation in the lungs early after RSV infection. In addition, the GCRR modulates inflammatory responses elicited by other TLR agonists, indicating that this sequence displays broad anti inflammatory properties.

Previous studies proposed a variety of immunological effects associated with the RSV Glycoprotein. Preferential priming with Glycoprotein, due to disruption of protein F during formalin inactivation of RSV, had been hypothesized to be the basis for an enhanced form of RSV disease that occurred in recipients of a formalin-inactivated RSV vaccine following subsequent natural exposure to RSV in the community²⁰. However, subsequent reports demonstrated that this protein is not necessary for disease enhancement by showing similar severity of illness after RSV challenge in mice immunized with inactivated RSV vaccines with or without Glycoprotein^{17,21}. In addition, the Glycoprotein has been reported to induce leukocyte chemotaxis in vitro¹⁹, and has been associated with an adaptive immune response that contributes to wheezing and asthma¹¹. The results reported herein show that RSV Glycoprotein inhibits production of inflammatory cytokines early after infection, thereby modulating the innate inflammatory response to the virus. These findings may have important implications for adaptive immunity. It is likely that Glycoprotein affects cytotoxic T lymphocyte responses and other mechanisms of viral clearance³, in addition to its effect on the Th bias of the immune response¹¹.

Several proteins and drugs modulate the innate inflammatory response. Glucocorticoids affect NF-κB dependent gene induction, presumably by interfering with direct contacts between p65 and the transcriptional machinery²². Some additional mechanisms that may silence NF-κB dependent genes in different cell lines are associated with the RBP Jκ co-repressor complex, Foxj1, the single immunoglobulin IL-1R-related molecule, and the p38 and ERK inhibitors^{23,24}. The wide specificity of GCRR modulation suggests that its effects are exerted directly or indirectly through pathways common to a variety of proinflammatory agents. Interestingly, the conserved GCRR has structural homology with the fourth subdomain of TNFR1, suggesting that its modulatory effect may be associated with binding and inactivation of TNF or an unknown TNF homologue²⁵. TNF also mediates endotoxin-induced shock, and TNFR1 deficient mice are resistant to lethal dosages of endotoxin^{26,27}. Furthermore, shedding of the TNFR1 modulates innate immune activation²⁸. Indeed, early secretion of RSV Glycoprotein may bind TNF-α and contribute to delay RSV clearance, as early production of TNF-α is protective against RSV infection in mice²⁹.

Sequencing studies have shown that the Glycoprotein is the most variable protein between the RSV subgroups, with only 53% identity between the proteins of subgroup A and B prototypical strains^{4,14}. The inhibitory effect described herein was characteristic of both RSV subgroups A and B and was elicited by the conserved GCRR, in which the cysteine residues forming two disulfide bonds between positions 173-186 and 176-182 (refs. 4,13) were required. Without being tied to one particular theory, because the amino acid sequences of the A and B GCCRs are not identical, this inhibitory effect may be associated with the conformation of the GCRR, rather than with the exact sequence. The conserved central region of the RSV Glycoprotein protein also contains a 13 amino acid segment that is immediately upstream of the GCRR and is conserved among human isolates.

The modulatory effect of the Glycoprotein on inflammation is also observed when using inactivated RSV. Therefore, the modulatory effect of the RSV Glycoprotein on NF-κB nuclear translocation is likely exerted by secreted Glycoprotein already present in supernatant fluids containing the RSV inoculum^{15,16,19}. Supporting this notion, reconstitution of cultures of human monocytes inoculated with mG with purified soluble Glycoprotein led to responses identical to those observed with wild type RSV. Interestingly, as infection in vivo is a sequential process involving multiple rounds of replication and spreading from infected to uninfected cells, the first cycle of RSV replication upon infection is probably not affected early on by the modulatory effect, but provides secreted Glycoprotein to areas where the virus is expanding and affects the immune response directed against it.

An important finding of these studies is that the GCRR can also modulate LPS-mediated cytokine production. Genetic polymorphisms in TLR4 but not in CD14 appear to affect severity of disease both during gram negative sepsis and RSV infection^{7,30}. The inhibitory effect of the GCRR on LPS-mediated inflammation may have implications for the treatment of severe diseases. LPS plays a critical role in many illnesses, among others septic shock due to gram-negative bacteria and development of childhood asthma^{30,31}. In addition, inflammation elicited by other pro inflammatory agents through other TLR receptors is also affected by this protein region.

In summary, this work identified a novel role for the RSV GCRR, a conserved domain present in all wild type isolates; provided new significance to the early secretion of Glycoprotein after RSV infection and revealed an increased complexity of the regulation of the host immune response during RSV infection.

### Example 7

### The RSV Glycoprotein is Critical for the RSV-Specific CTL Response

The cytotoxic T lymphocyte (CTL) response plays an important role in the control of replication of a wide variety of viruses¹⁷. CD8⁺ T cells recognize MHC class I molecules carrying 8-10 amino acid-long peptides and control infection by direct destruction of infected cells or by the release of antiviral cytokines¹⁷. In infections caused by RSV, the CD8⁺ T cells appear to play an important role in protective immunity and recovery from infection 18-20⁴⁻⁶. In addition, RSV-specific CTLs are critical for Th1 skewing of the CD4⁺ T cell response after vaccination²¹⁻²³. Th1 skewing is presumed to be desirable for the development of safe vaccines against RSV, because a Th2 bias of the immune response was linked to a severe form of RSV disease in recipients of a formalin-inactivated RSV vaccine subsequently exposed to wild type virus in the 1960s²¹⁻²⁶.

The BALB/c mouse is widely used as a model for study of RSV infection. The dominant RSV-specific CTL epitope for BALB/c mice is encoded between positions 82 and 90 of the anti-termination factor M2-1 (M2⁸²⁻⁹⁰)²⁷⁻³⁰. This H2-K^{d} restricted epitope is estimated to encompass ˜40% of the primary RSV-specific H-2^{d} restricted CTL response³¹. A subdominant CTL epitope in H-2^{d} mice is located in the main neutralization antigen of RSV, the fusion protein (F), positions 85-93 (F⁸⁵⁻⁹³), and is responsible for <5% of the primary antiviral CTL response 32,33^{18,19}. Conversely, the other neutralization antigen in RSV, the attachment protein (RSV Glycoprotein), lacks H-2^{d} restricted epitopes^{21,22,27,29}. Furthermore, unlike most other RSV proteins, RSV Glycoprotein has not been demonstrated to elicit CTL activity in humans^{27,34-36}.

The RSV Glycoprotein is produced as a transmembrane form with a cytoplasmic tail and a proximal hydrophobic signal anchor, and as a truncated soluble form that is rapidly secreted^{16,37}. The ectodomain of the RSV Glycoprotein includes two mucin-like segments, with divergent amino acid sequences between isolates, and a short, circumscribed central region that is highly conserved between RSV antigenic subgroups A and B 38²⁴. This conserved region includes four cysteine residues (positions 173,176,182 and 186) that form a cystine noose held by disulfide bonds between Cys¹⁷³ and Cys¹⁸⁶, and between Cys¹⁷⁶ and Cys¹⁸². The RSV G cysteine-rich region (GCRR) originally was speculated to play a role in receptor binding¹⁶, but recent data have shown that it is not required for efficient infection in vitro and in mice^{39,40}. However, the GCRR can modulate inflammation by inhibiting Toll-like receptor 4 (TLR4) activation and NF-κB nuclear translocation⁴¹. And recent publications suggested a role for TLR4 in RSV clearance from infected lungs^{42,43}. Therefore, we speculated that the GCRR might also affect the CTL response against RSV.

As reported in more detail below, a novel and unexpected function for the GCRR is herein identified. Despite lacking H-2^{d} restricted epitopes, the RSV GCRR is required for the generation of a CTL response during RSV infection. These findings are relevant to the understanding of mechanisms of cell-mediated immunity against RSV and may contribute to the design of new candidate vaccines.

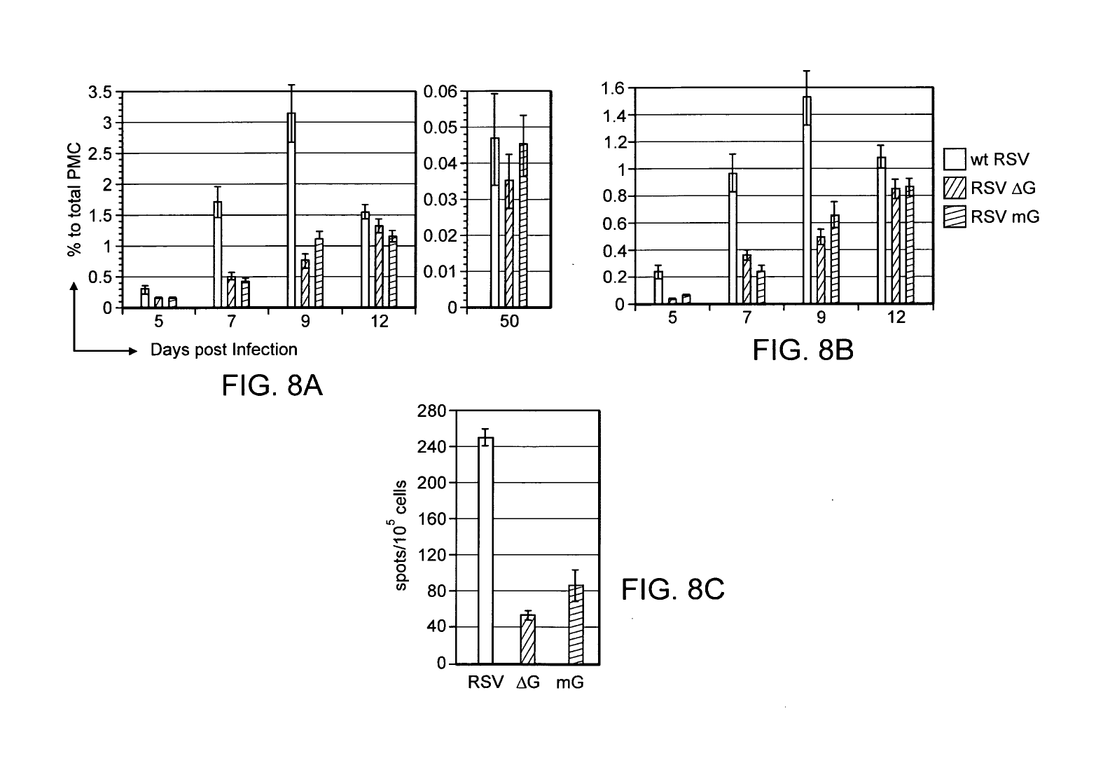

To determine the role of RSV Glycoprotein in the RSV-specific CTL response, the numbers of RSV-specific CTL were compared in mice after intranasal infection with wild-type RSV, a recombinant RSV that lacks the entire RSV Glycoprotein gene (ΔG), and a recombinant RSV that expresses only the membrane bound, but not the secreted form of the RSV Glycoprotein (mG). At specified days post-infection, PMC were isolated and analyzed by three different methods. First, the number of RSV-specific CD8⁺ T cells were quantitated by staining of PMC with anti-CD8 antibody and a tetramer specific to the RSV M2⁸²⁻⁹⁰ immunodominant epitope (FIG. 8A). Second, to quantitate the T cells secreting IFN-γ in response to specific stimulation, PMC were stimulated with the RSV M2⁸²⁻⁹⁰ CTL immunodominant peptide, stained for CD8 and IFN-γ, and analyzed by flow cytometry (FIG. 8B). Third, the number of cells secreting IFN-γ in response to specific stimulation was determined by immunospot assay (FIG. 8C). The virus-specific CD8⁺ T cell response induced by wild-type RSV was detectable by flow cytometry at 5 days and peaked 9 days after infection (FIGS. 8A and 8B). The number of RSV-specific CTL induced by ΔG and mG was significantly lower at all time points. The kinetics of the CTL response elicited by the two viruses lacking one or both forms of RSV Glycoprotein were delayed and peaked 12 days after infection, always at lower levels than the response induced by wild-type RSV. This observation suggested that the secreted form of the RSV Glycoprotein protein is necessary for eliciting an effective RSV-specific CTL response. As previously described for wild-type RSV³¹, on day 50, the responses evaluated by tetramer staining decreased to very low levels for all viruses (FIG. 8a), and were not detectable by IFN-γ staining.

To determine whether the differences in CTL numbers evaluated by tetramer and IFN-y staining were associated with differences in cytolytic activity, the levels of cytolysis were evaluated ex vivo by measuring the release of lactate dehydrogenase (LDH) from RSV-infected and M2⁸²⁻⁹⁰-specific target cells exposed to PMC of infected mice (FIG. 9). Again, cytolytic activity was greater in mice infected with wild-type RSV than in animals infected with ΔG or mG (P<0.01).

### Example 8

### Co-Administration of RSV Glycoprotein During Infection can Enhance the CTL Response

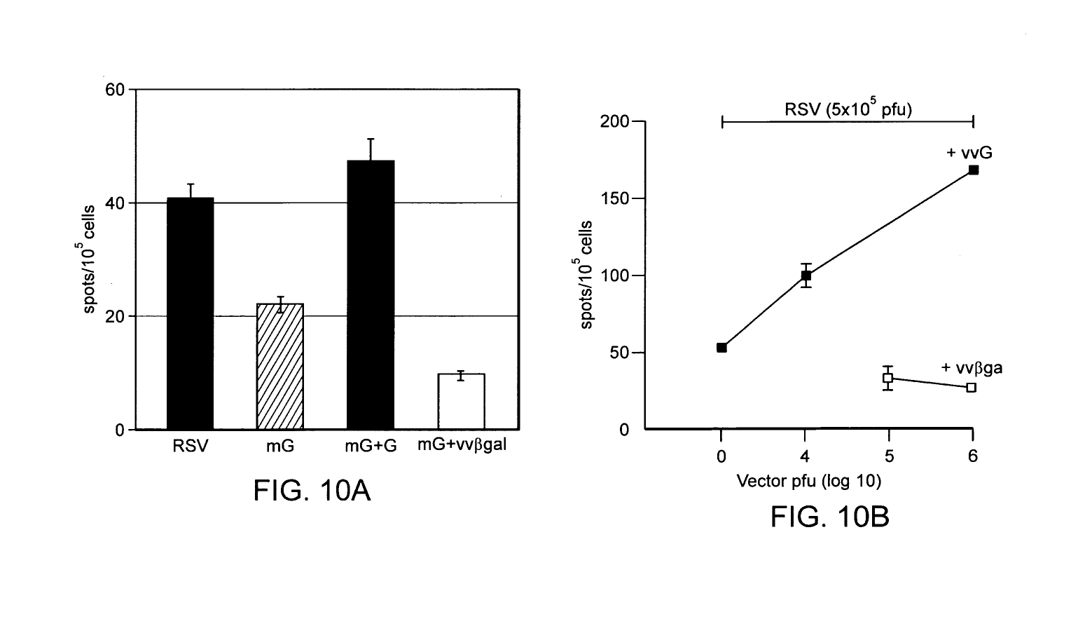

To determine whether the simultaneous administration of a vector expressing RSV Glycoprotein with the Glycoprotein-deficient recombinant viruses would re-establish a RSV-specific CTL response of similar magnitude as that elicited by wild-type RSV infection, mice were infected with wild-type RSV, mG alone, or mG with a recombinant vaccinia virus expressing the RSV G gene (vvG) or an irrelevant control gene (vvβgal). Interestingly, M2⁸²⁻⁹⁰ specific CTL activity was restored by co-administration of wG with mG to levels similar to those detected after infection with wild-type RSV (FIG. 10A). Conversely, co-administration of vvβgal and mG resulted in low responses, similar to those observed after infection with mG alone. These findings support a role for Glycoprotein in promoting RSV-specific CTL responses during infection.

To determine whether incremental addition of Glycoprotein to wild-type RSV infection could further increase the RSV-specific CTL response in a dose-dependent manner (FIG. 10B), mice were inoculated with wild-type RSV and incremental doses of vvG or vvf3gal. Interestingly, addition of vvG (at all doses tested) increased the RSV-specific CTL response. This enhancement was dose-dependent and suggested that physiologic anti-RSV CTL responses are further enhanced by addition of RSV Glycoprotein.

### Example 9

### Differences in CTL Activity are not Explained by Differences in Pulmonary Replication or Differences in the Inflammatory Response

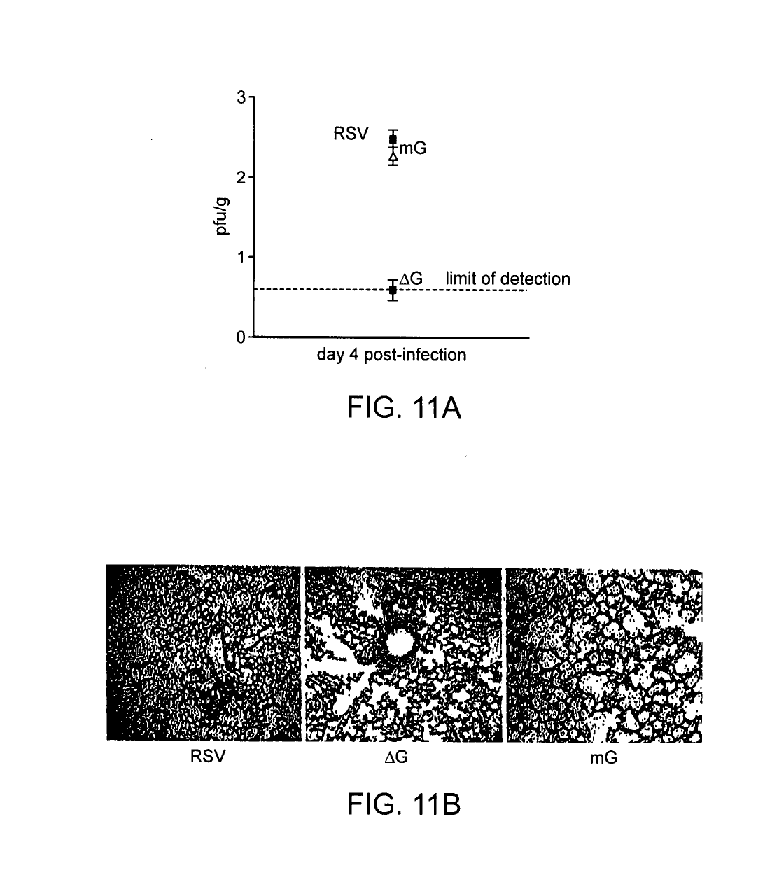

The magnitude of the CTL response is often determined by the virus titer during infection. Therefore, to examine whether the differences in CTL response were associated with differences in replication, virus titers in lungs after infection with wild-type RSV or the recombinant viruses lacking one or both forms of G (FIG. 11A) were compared. Even though wild-type RSV and mG elicited significantly different CTL responses (wt RSV>mG; see FIGS. 8 and 9), both viruses replicated to similar titers, while replication of ΔG was further reduced and was detectable only in ⅖ infected animals. However, the CTL response elicited by ΔG was similar to that of mG recipients. These findings demonstrated that the differences observed in the magnitude and lytic capacity of CTL responses elicited by the different RSV constructs cannot be explained by their respective viral titers.

In addition, G-deficient viruses promoted significant innate inflammation in the lungs of mice early (24 h.) after inoculation⁴¹. Without being tied to one particular theory, a possible explanation for the poor CTL response detected in mice infected by these viruses may be a relative excess of pulmonary macrophages over lymphocytes in these samples, compared to those obtained from wild-type RSV recipients. Alternatively, the relative excess of macrophages could decrease the number of CD8⁺ T cells in the total cells selected for the assays. As previously reported⁴¹, and unlike twenty-four hours post-infection, no differences between the groups in differential counts from pulmonary infiltrates, as determined by histopathology were observed seven days after infection (FIG. 11B). Due to the relatively low dose of the inoculum (5×10⁵ pfu), only mild perivascular and peribronchiolar granulocytic and mononuclear cellular infiltration with mild alveolitis was present in all groups.

### Example 10

### The Conserved GCRR is Necessary to Elicit RSV-Specific Cytotoxicity

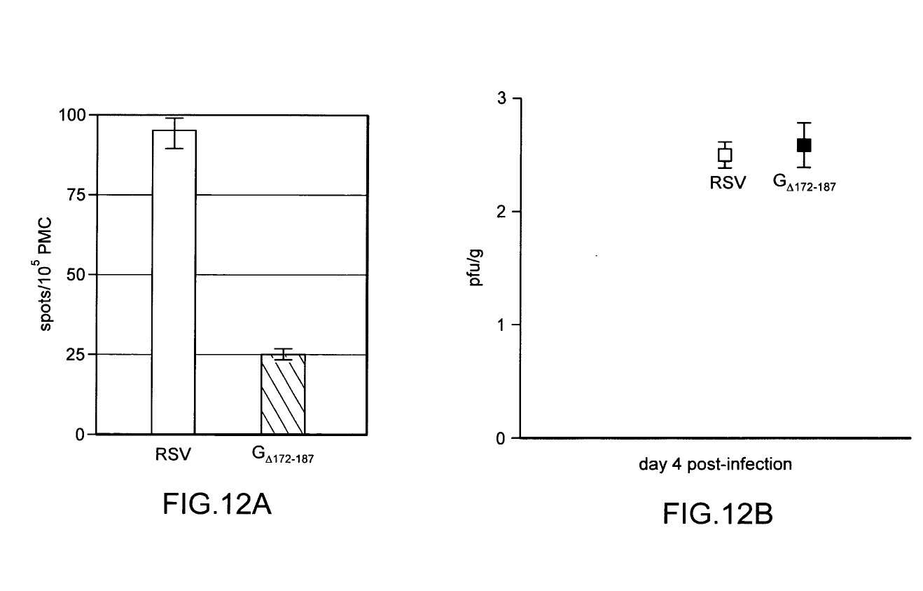

Recently, the conserved GCRR was shown to have immune modulatory properties during RSV infection⁴¹. Therefore, to determine whether the GCRR could affect CTL activity the M2⁸²⁻⁹⁰ specific CTL response elicited by wild-type RSV and the recombinant RSV lacking the GCRR (ΔG₁₇₂₋₁₈₇) was compared. For this purpose, an immunospot assay was used to quantitate the number of IFN-γ-positive cells (FIG. 12A). Even though ΔG₁₇₂₋₁₈₇ viral titers in the lungs were similar to those of wild-type RSV (FIG. 12B), the CTL response in PMC from mice infected with ΔG₁₇₂₋₁₈₇ was significantly lower than the response observed after wild-type RSV infection (FIG. 12A). These results were similar to those previously observed after infection with other G-deficient viruses (FIGS. 8 and 9), and suggested that the GCRR is required for the production of an effective CTL response against RSV.

These studies describe a novel role for the RSV Glycoprotein and its GCRR. Despite lacking H-2^{d} restricted epitopes, RSV Glycoprotein is necessary for the development of an effective RSV-specific CTL response during primary infection. This pro-CTL effect is associated, at least in part, with a widely conserved central segment of the protein, the GCRR. Infection with mG, a recombinant virus that does not express the secreted form of Glycoprotein (including its GCRR), resulted in a significantly reduced CTL response. Therefore, it is likely that secretion of G with its GCRR is necessary for the generation of RSV-specific cytotoxicity.

The CTL response is important for control of RSV replication in the respiratory tract. Depletion of CD8⁺ T cells in mice results in elevated virus titers seven days after infection and delayed pulmonary clearance²⁰. Furthermore, immunization of mice with a recombinant vaccinia virus expressing the RSV immunodominant H-2^{d} restricted epitope encoded in M2⁸²⁻⁹⁰ conferred transient protection against RSV challenge²⁹. However, the duration of cell-mediated immunity against RSV is limited, and this may constitute a problem for RSV vaccine development. This may be overcome by co-administration of GCRR with RSV. IFN-γ played a crucial role in CTL-mediated RSV clearance from the lungs, while deficiencies in perforin or Fas-L do not appear to affect the peak or duration of pulmonary replication 44³⁰.

The GCRR pro-CTL effect modifies a long-standing paradigm in RSV immunology: the idea that G is not necessary for the generation of a CTL response against RSV. In fact, the enhanced pulmonary disease that affected recipients of a formalin-inactivated RSV vaccine in the 1960s was thought to be associated, at least in part, with a Th2 polarization of pulmonary T cells resulting from the absence of a CTL response after vaccination²¹⁻²³. This poor CTL response has long been associated with the disruption of RSV F epitopes during formalin inactivation, creating an imbalance in the vaccine in favor of Glycoprotein, a protein without CTL activity²¹⁻²³. The present studies suggested that despite the apparent absence of mouse or human CTL epitopes in RSV G, the protein plays a critical role in the induction of cytotoxicity.

The observation that supplementation with RSV G can enhance the specific CTL response in a dose-dependent manner beyond its natural level is important for vaccine design. Incorporation of additional Glycoprotein genes to recombinant viruses and/or relocation of the gene upstream in the viral genome to enhance its transcription are strategies that will likely improve the cellular response against RSV. The benefit of enhancing the RSV-specific CTL response should be weighted against other potential modulatory properties of the GCRR⁴¹ and other regions of RSV Glycoprotein^{21,22,25,47}. The mechanism of the pro-CTL effect of the GCRR is intriguing. It is not clear that any other viral proteins that, lacking MHC class I-restricted epitopes, promote virus-specific cytotoxicity have been previously reported. This effect, however, controls virus clearance, and therefore is probably an inescapable trade off from a different beneficial effect for the virus that is elicited by the GCRR. For instance, the GCRR modulates the production of inflammatory cytokines mediated by TLR4 early after infection⁴¹. Modulating TLR4 may play a role in the pathogenesis of RSV, as infants with loss-of-function single nucleotide polymorphisms in TLR4 are epidemiologically associated with increased severity of illness and decreased oxygen saturation⁴⁵. This TLR4 modulation affects production of interleukin (IL)-10, a cytokine involved in the modulation of CTL responses in other models^{41,46}. A second potential explanation for the observed effect may be associated with the fractalkine motif encompassed in the GCRR between amino acids 182 and 186, which is also disrupted in ΔG₁₇₂₋₁₈₇. Fractalkine may enhance CTL activity through chemoattraction and activation of dendritic cells^{48,49}. Finally, the anti inflammatory effect of the GCRR may affect the number of antigen presenting cells⁵⁰.

In summary, a novel and unexpected role for the cysteine-rich region of RSV G is described herein. This positive modulatory effect on CTL function may be important for RSV vaccine design. Furthermore, the GCRR may be useful for eliciting a broader beneficial effect on protective CTL responses against other illnesses.

### Example 11

### RSV G Increased the Cytotoxic T Lymphocyte (CTL) Response During Malaria Infection

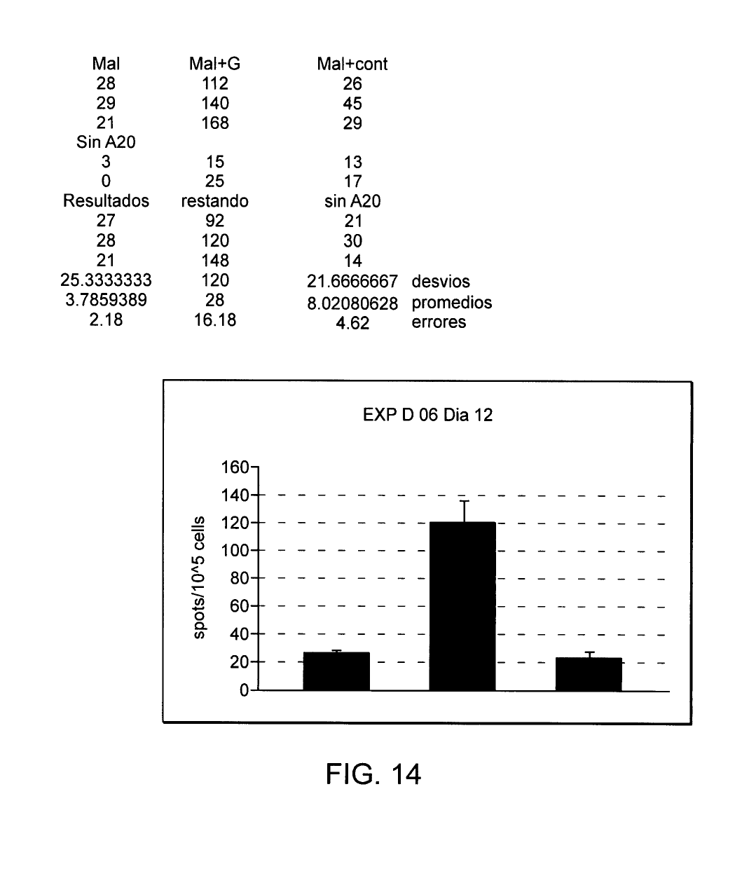

Mice innoculated with malaria and a vector encoding the cysteine-rich region of RSV G showed an enhanced immune response relative to control mice infected with malaria alone or malaria and a control vector encoding a scrambled peptide. FIG. 14 shows that malaria infected mice co-injected with the cysteine-rich region of RSV G showed a dramatic increase in the cytotoxic T lymphocyte (CTL) response.

### Example 12

### RSV Glycoprotein Increase the Cytotoxic T-Cell Response Against Influenza Virus

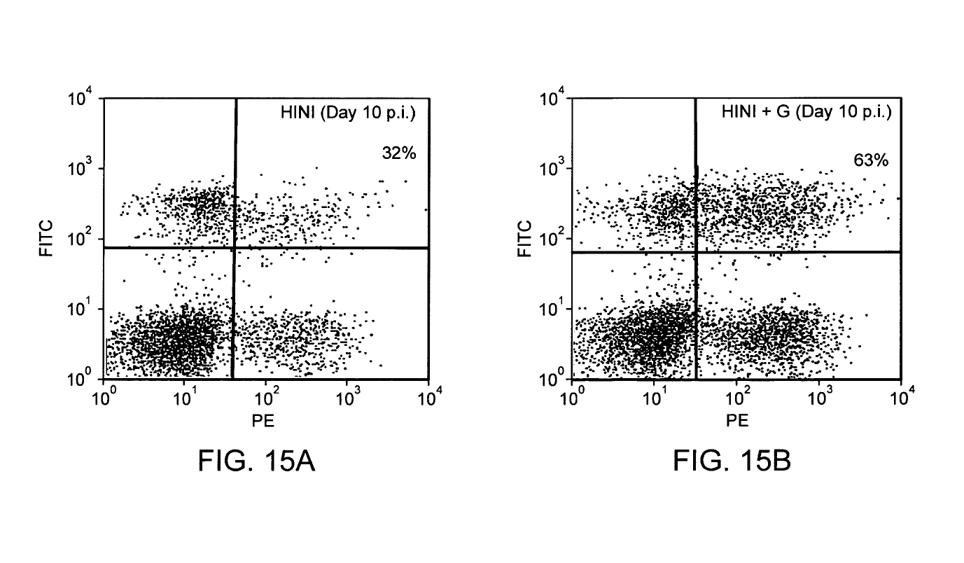

The cytotoxic response by T-lymphocytes (CTL) is critical for the clearance of influenza virus infection. Improving CTL against influenza is therefore desirable. Animal models in C57BL/6 mice have shown that two major viral epitopes co-dominate the immune response during primary infection against influenza. These peptides are located in the viral nucleoprotein (NP₃₆₆₋₃₇₄/D^{b}) and acidic polymerase (PA₂₂₄₋₂₃₃/D^{b}). The RSV attachment glycoprotein (RSV-G) lacks H2-b restricted epitopes and has not been shown to elicit CTL responses in mice and humans. However, this protein enhances the CTL activity against epitopes in other RSV proteins in a dose-dependent manner. To determine whether RSV-G can elicit a similar positive regulatory effect during a heterologous infection with influenza virus, mice co-immunized with H1N1 or H3N2 and a DNA construct encoding RSV-G displayed enhanced cytotoxic T-cell activity against NP₃₆₆₋₃₇₄ and PA₂₂₄₋₂₃₃ on days 6/7, 10 and 14 postinfection compared to mice infected only with influenza virus or with influenza virus and a DNA construct encoding an irrelevant gene, as determined by IFN-γ production (ELISPOT). Results were confirmed on a per cell lytic activity by granzyme-B expression (flow cytometry) as shown in FIGS. 15A and 15B. These findings indicate that co-administration of vectors encoding RSV-G can play an important role for the design of novel immunization strategies against other respiratory pathogens.

These experiments were carried out using the following materials and methods.

Virus Infection and Sampling.

4-6 week old female C57BL/10 mice (Jackson Laboratories, Bar Harbor, Me.) were used for these experiments. Intranasal infection was performed with 10⁶ pfu of live RSV, ΔG and mG. RSV titers in the lungs of mice were determined as previously described¹⁷. A severity scoring system was used to characterize the degree of pulmonary infiltration. Briefly, for innate inflammation the lung parenchyma was scored as: 0⁼absence of inflammation; 1=less than 20% of field with focal polymorphonuclear (PMN) and macrophage inflammation; 2=20% or more of the field with focal PMN and macrophage inflammation; and 3=diffuse PMN and macrophage inflammation. Scores were assigned by blinded examiners (5-6 mice/group).

Monocyte Stimulation.

Human PBMC were isolated from leukopaks using Histopaque (Sigma, St. Louis, Mo.). Monocytes were isolated using the Monocyte Isolation Kit II (positive selection for CD3, CD7, CD16, CD19, CD56, CD123, and glycophorin A, MiltenyiBiotec) with Macs LS separation columns. Remaining cells were >90% monocytes by anti-CD14 staining and forward- and side-light scatter analysis using FACScan (Becton-Dickinson, Elmhust, Ill.).

Purified monocytes were stimulated with LPS (1 μg) or purified proteins F or Glycoprotein (3 μg; kindly provided by V. Randolph, Wyeth Lederle, N.Y.). All rRSV variants were grown in Hep-2 cells and Vero cells as described^{29,30}, and used at 10⁵ pfu multiplicity of infection (MOI)=1] for stimulations. Lysate of Hep-2 cells and Vero cells were used as control stimuli. UV-inactivation was performed as described elsewhere19⁵ and lack of virus replication confirmed in cell culture. For additional experiments, three RSV (G_{Cys182Arg}; G_{Cys186Arg} and control) selected using Glycoprotein-specific monoclonal antibodies as described were used28¹⁴. Peptides used for stimulations included the GCRR¹⁶⁴HFEVFNFVPCSICSNNPTCWAICKRI¹⁸⁹ [SEQ ID NO:______], a cysteine to serine control peptide¹⁶⁴HFEVFNFVPSSISSNNPTSWAISKRI¹⁸⁹ (GSRR) [SEQ ID NO:______], and a³²¹YFARGPGIHIRKR³⁰⁷ [SEQ ID NO:______] reverse-oriented HIV V3 loop. An additional peptide encoding the conserved 13 amino acid region upstream of the GCRR (164-176)³ was also used in add-back experiments. All peptides were synthesized by 9-fluorenylmethoxycarbonyl solid-phase chemistry (SynPep, Dublin, Calif.). Selective formation of disulfide bonds in GCRR was accomplished by protection of two selective cysteine residues (176, 182) with acid-labile groups, and two with non-acid labile groups (173,186). Sequential removal of the acid-labile protection groups followed by oxidation and disulfide bond formation, and subsequent deprotection of non-acid labile groups followed by the same process led to selective formation of the native 1-4/2-3 bridges. Peptides were tested by analytical HPLC, mass spectrometry and LC/MS (SynPep, Dublin, Calif.). The GCRR and control peptides included biotin-SGSG at the amino termini. The anti-CX3CR1 antibody was kindly provided by P. Murphy, NIAID, NIH and used as described¹⁸. For supplemental experiments, we used peptidoglycan (PGN; Fluka, Sigma) at 10 μg and CpGDNA (GTCGTT; HyCult Biotechnology) at 1 mM. Cytokines were measured in supernatant fluids 18 hours after stimulation by immunoassay following manufacturer's instructions (Biosource Europe S. A, Belgium).

Flow Cytometry.

Alveolar macrophages were obtained by bronchoalveolar lavage (BAL) followed by magentic bead depletion (Militenyi Biotec, Germany). Macrophages were incubated with brefeldin A for six hours, fixed with commercially available fixation and permeabilization reagents, CYTOFIX/CYTOPERM (Becton Dickinson, Elmhust, Ill.), and stained using phycoerythrin (PE)-conjugated anti-IL-6 antibody (Becton Dickinson). Data was analyzed using side and forward scatter plots and FACScan (Becton Dickinson).

NF-κB Nuclear Translocation Immunoassay.

Human monocytes were stimulated with purified RSV F and/or Glycoprotein (1 μg each) or indicated viruses for 60 minutes. After stimulation nuclear extracts were obtained using a hypotonic lysis buffer (10 mM HEPES (pH 7.9), 1.5 mM MgCl₂, 10 mM KCl, 0.5 mM DTT, 0.1% Triton X-100 and protease inhibitors) and an extraction buffer (20 mM HEPES (pH 7.9), 1.5 mM MgCl₂, 0.42 M NaCl, 0.5 mM DTT, 0.2 mM EDTA, 1.0% Igepal CA-630, 25% (v/v) glycerol and protease inhibitors). NF-κB subunits p50 and p65 were detected by a modified immunoassay using a double stranded biotinylated oligonucleotide containing the consensus sequence for NF-κB binding (5′-GGGACTTTCC-3′) [SEQ ID NO:______] (Chemicon International, Germany).

I_{κ}B_{α} Western Blot.

Purified human monocytes were incubated with the corresponding recombinant RSV for 60 minutes at 37° C., collected and lysed in Isotonic Buffer (10 mM Hepes-KOH [pH [7.2], 142.5 mM KCl, 5 mM MgCl₂, 1 mM EGTA, 0.3% NP-40). Proteins were separated by SDS/PAGE, transferred onto PVD membranes (Millipore, Bedford, Mass.), and blocked with 5% milk in PBS-T (1×PBS, 0.1% Tween-20). I_{κ}B_{α} was detected with a rabbit anti-I_{κ}B_{α} (Santa Cruz Biotechnology, Santa Cruz, Calif.), followed by a HRP-conjugated anti-rabbit IgG (Amersham Corp, Arlington Heights, Ill.) and developed with a commercially available chemiluminescent substrate, SUPERSIGNAL PICO CHEMILUMINESCENT SUBSTRATE (Pierce, Rockford, Ill.).

Statistical Analysis.

Data were analyzed with statistical software (Statview). Comparisons were made using the Mann Whitney U test where appropriate. All experimentation was approved by The Johns Hopkins Medical Institutions.

Virus Infection and Sampling.

4-10 weeks-old female BALB/c mice (The Jackson Laboratory, Bal Harbor, Me.) were housed under laminar flow hoods in an environmentally controlled specific pathogen-free animal facility. Intranasal infections were performed with 5×10⁵ pfu of live wild type RSV, or the following recombinant RSVs: lacking the entire Glycoprotein gene (ΔG); expressing only the membrane but not the secreted form of the Glycoprotein protein (mG); lacking the GCRR (ΔG₁₇₂₋₁₈₇)^{39,40}. All experimentation was approved by and performed according to guidelines of the Johns Hopkins Medical Institutions and the National Institutes of Health.

Flow Cytometry.

Isolation of pulmonary mononuclear cells (PMC), intracellular staining of interferon-γ (IFN-γ) and flow cytometry were performed as described⁵⁰. For quantitation of RSV-specific CTL, lung PMC were isolated from mice⁵⁰, washed twice in phosphate buffered saline (PBS) containing 2% fetal bovine serum (PBS), and stained with an optimized amounts of phycoerythrin (PE)-conjugated MHC class I H-2K^{d} tetramer complexes loaded with the peptide SYIGSINNI (NIAID Tetramer Facility, Yerkes Regional Primate Research Center, Atlanta, Ga.), representing the immunodominant epitope of the RSV M2-1 protein^{30 16}, and fluorescein isothiocyanate (FITC)-conjugated rat anti-mouse CD8α monoclonal antibody, clone 53-6.7 (BD Biosciences).

To analyze the cells that secrete IFNγ in response to RSV-specific stimulation, PMC were resuspended in RPMI medium 1640 (Invitrogen, Carlsbad, Calif.) containing 10% FBS, 100 U of penicillin/ml and 100 μg of streptomycin sulfate/ml. The cells were counted and incubated overnight with 1 μM of the M2-1 peptide in the presence of GolgiStop (Invitrogen) protein transport inhibitor monensin. After stimulation, cells were washed twice with PBS containing 2% FBS, treated with FC BLOCK (BD Biosciences), which is a purified rat IgG_{2b}anti-mouse CD16/CD32 monoclonal antibody, to block Fc receptors, stained as described above with FITC-conjugated anti-mouse CD8α monoclonal antibody, washed twice, fixed and permeabilized with Cytofix/Cytoperm Solution (BD Biosciences). This was followed by staining with allophycocyanin (APC)-conjugated rat anti-mouse IFNγ antibody, clone XMG1.2 (BD Biosciences). Flow cytometry analysis was performed using a FACSCALIBUR FLOW CYTOMETER (BD Biosciences). A total of 30,000 cells were analyzed per sample.

Immunospot Assay.

Nitrocellulose-based 96-well microtiter plates (Milliliter HA, Millipore, Bedford, Mass.) were coated overnight at room temperature with 10 μg/ml of anti-IFN-γ monoclonal antibody (clone R4-6A2, BD Biosciences). PMC were incubated in the coated plates for 18 hours with irradiated target A-20 B cell lymphoma line (American Type Culture Collection, Manassas, Va.) loaded with the M2⁸²⁻⁹⁰ peptide. Spots corresponding to individual IFN-γ producing cells were revealed with biotinylated anti-IFN-γ monoclonal antibody (clone XMG1.2, BD Biosciences) followed by streptavidin peroxidase and 3,3′-diaminobenzidina tetrahydrochloride dehydrate (Sigma, Saint Louis, Mo.). All assays were performed in triplicates.

Cytolytic Activity.

A standard cytolytic assay was performed using RSV-infected and uninfected, or the M2⁸²⁻⁹⁰ peptide-loaded A-20 target cells. Target cells were incubated with effector PMC in a top effector-target ratio of 50:1 in V-bottom plates (Costar). Plates were centrifuged for 30 seconds. at 150×G prior to a 6 hour incubation at 37° C. in 5% CO₂. Cells were gently pelleted and 100 μl of supernatant fluid transferred for determination of released lactose dehydrogenase (LDH) according to the manufacturer's instructions (Cytotoxicity Detection Kit, Roche, Indianapolis, Ind.). Percent specific lysis was calculated as previously described³³.

RSV Titers in the Lungs.

Lungs from mice were removed aseptically and ground in 3 ml of buffer, HANKS MEDIA (Invitrogen). Debris was pelleted by centrifugation and samples were plated on Vero cells. Monolayers were then overlaid with Opti-MEM cell culture medium (Invitrogen) with 2% fetal calf serum, 0.8% methylcellulose, glutamine and antibiotics and incubated for 5 days. Plates were stained by the immunoperoxidase method and results expressed in pfu/g.

Histopathology.

Lungs from mice were removed 4 and 7 days after challenge, fixed overnight with 10% buffered formalin at 4° C. and embedded in paraffin. Lung sections were stained with periodic acid schiff (PAS) reaction to examine the inflammatory infiltration. Briefly, to characterize the pneumonia the vessels and bronchi were scored, with scores ranging from 1 to 3, where a score of 1 denotes that the tissue is free from or with few infiltrating cells; 2 denotes the presence of focal aggregates of infiltrating cells or the structure cuffed by one definite layer of infiltrating cells; 3, with two or more definite layers of infiltrating cells with or without focal aggregates⁵¹. Subsequently, the histopathology was categorized as mild (>60% with score=1; 0% with score=3), moderate (>30% score=2; <20% score=3) or severe (>20% score=3).

Statistical Analysis. Data were Analyzed with Statistical Software (Statview). Comparisons Were Made Using the Mann Whitney U Test where Appropriate.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

### Other Embodiments

From the foregoing description, it will be apparent that variations and modifications may be made to the invention described herein to adopt it to various usages and conditions. Such embodiments are also within the scope of the following claims. All patents and publications mentioned in this specification are herein incorporated by reference to the same extent as if each independent patent and publication was specifically and individually indicated to be incorporated by reference.

### REFERENCES

1. Kurt-Jones et al., (2000) Nat. Immunol. 1, 398-401
2. Haynes et al., (2001) J Virol 75, 10730-7
3. Collins, et al., (2001) Fields Virology 1443-1486
4. Haeberle, et al., (2002) J infect Dis. 186, 1199-206
5. Hull, et al., (2000) Thorax. 55, 1023-7
6. Tal, et al., (2004) J Infect Dis. 189, 2057-63
7. Chang, et al., (2001) J. Immunol. 167, 4254-60
8. Nicholas, et al., (1990) J Virol. 64, 4232-41
9. Jackson, et al., (1996) J Med Virol 49, 161-9
10. Hancock, et al., (200111) J Infect Dis. 184, 1589-93
11. Tripp, et al., (2000) J Virol. 74, 6227-9
12. Johnson, et al., (1987) Proc. Natl. Acad. Sci. 84:5625
13. Rueda, et al., (1994) Virology, 198; 653-662
14. Teng, et al., (2002) J Virol. 76, 6164-71
15. Teng, et al., (2001) Virology 289, 283-96
16. Conlins, et al., (2001) Virology 1443-1486
17. Russell, et al., (2002) Annu. Rev. Immunol. 20: 323-370
18. Cannon, et al., (1988) J. Exp. Med. 168: 1163-1168
19. Cannon, et al., (1987) Immunology. 62: 133-138
20. Graham, et al., (1991) J. Clin. Invest. 88: 1026-1033
21. Srikiatkhachorn, et al., (1997) J. Exp. Med. 186(3): 421-432
22. Hussell, et al., (1997) Eur. J. Immunol. 27: 3341-3349
23. Tang, et al., (1994) RSV. J. Clin. Invest. 94: 1953-1958
24. Connors, et al., (1992) J. Virol. 66: 7444-7451
25. Waris, et al., (1996) J. Virol. 71: 2852-2860
26. Graham, et al. (1993) J. Immunol. 151: 2032-2040
27. Nicholas, et al., (1990) J. Virol. 64: 4232-4241
28. Openshaw, et al., (1990) J. Virol. 64: 1683-1689
29. Connors, et al., (1992) J. Virol. 66: 1277-1281
30. Kulkarni, et al., (1995) J. Virol. 69: 1261-1264
31. Chang, et al., (2002) Nat. Med. 8: 54-59
32. Jiang, et al., (2002) J Gen Virol. 83: 429-438
33. Chang, et al., (2001) J Immunol. 167: 4254-4260
34. Heideman, et al. (2004) J Gen Virol. 85: 2365-2374
35. Rock, et al., (2003) Immunology 108: 474-80
36. Venter, et al., (2003) J Virol. 77: 7319-7329
37. Melero, et al., (1997) J Gen Virol. 78: 2411-2418
38. Martinez, et al., (1997) J Gen Virol., 78: 2419-2429
39. Teng, et al., (2002) J Virol. 76: 6164-6171
40. Teng, et al., (2001) Virology. 289: 283-296
41. Polack, et al., (2005) Submitted for publication
42. Haynes, et al., (2001) J Virol 75(22): 10730-10737
43. Kurt-Jones, et al., (2000) Nat Immunol. 1(5): 398-401
44. Oster, et al., (2002) Eur. J. Immunol. 32: 2117-2123
45. Tal, et al., (2004) J Infect Dis. 189: 2057-2063
46. Kurte, et al., (2004) J Immunol. 173: 1731-7
47. Tripp, et al., (2001) Nat Immunol. 2:732-8
48. Guo, et al., (2003) Int J Cancer 103: 212-20
49. Niess, et al., (2005) Science. 307: 254-8
50. Bukreyev, et al., (2001) J Virol 75: 12128-40
51. Connors, et al., (1992) J. Virol. 66: 7444-6451

1. An isolated RSV Glycoprotein fragment having immunomodulatory activity.

2. The RSV Glycoprotein fragment of claim 1, wherein the fragment comprises a sequence selected from the group consisting of:
cysteines at an amino acid position corresponding to cysteines 182 and 186 of human RSV;
at least four cysteine residues corresponding to cysteines 173, 176, 182, and 186;
at least a Glycoprotein cysteine rich region (GCRR);
at least amino acids 164-189 of the RSV Glycoprotein; and
at least amino acids 173-186 of an RSV Glycoprotein.

3-9. (canceled)

10. The RSV Glycoprotein fragment of claim 1, wherein the fragment is a human, bovine, or ovine RSV Glycoprotein.

11-15. (canceled)

16. An isolated RSV Glycoprotein nucleic acid molecule encoding the fragment of claim 1.

17. A vector comprising an RSV Glycoprotein nucleic acid molecule encoding a polypeptide of claim 1.

18-22. (canceled)

23. A viral vector comprising an RSV Glycoprotein nucleic acid molecule encoding a polypeptide of claim 1.

24-34. (canceled)

35. A pharmaceutical composition comprising an effective amount of an RSV Glycoprotein fragment that comprises a sequence selected from the group consisting of:
cysteines at an amino acid position corresponding to cysteines 182 and 186 of human RSV;
at least four cysteine residues corresponding to cysteines 173, 176, 182, and 186;
at least a Glycoprotein cysteine rich region (GCRR);
at least amino acids 164-189 of the RSV Glycoprotein; and
at least amino acids 173-186 of an RSV Glycoprotein
in a pharmaceutically acceptable excipient, wherein the fragment is capable of modulating an immune response in a subject.

36-45. (canceled)

46. A pharmaceutical composition comprising an effective amount of a nucleic acid molecule encoding an RSV Glycoprotein fragment of claim 1 in a pharmaceutically acceptable excipient, wherein the fragment is capable of modulating an immune response in a subject.

47-49. (canceled)

50. A pharmaceutical composition comprising an effective amount of a viral vector of claim 23.

51. An immunogenic composition comprising an RSV Glycoprotein fragment in a pharmaceutically acceptable excipient.

52-54. (canceled)

55. A method of modulating an immune response in a subject in need thereof, the method comprising administering to the subject an RSV Glycoprotein fragment capable of modulating an immune response or a polynucleotide encoding the fragment.

56-57. (canceled)

58. A method of decreasing a Toll-like receptor (TLR) function in a subject in need thereof, the method comprising administering to the subject an RSV Glycoprotein fragment capable of modulating an immune response or a polynucleotide encoding the fragment.

59-60. (canceled)

61. A method of decreasing an inflammatory response in a subject in need thereof, the method comprising administering to the subject an RSV Glycoprotein fragment capable of modulating an immune response comprising a sequence selected from the group consisting of:
cysteines at an amino acid position corresponding to cysteines 182 and 186 of human RSV;
at least four cysteine residues corresponding to cysteines 173, 176, 182, and 186;
at least a GCRR;
at least amino acids 164-189 of the RSV Glycoprotein; and
at least amino acids 173-186 of an RSV Glycoprotein
or a polynucleotide encoding the fragment.

62-81. (canceled)

82. A method of enhancing an immune response in a subject against an immunogenic composition, the method comprising administering an effective amount of a pharmaceutical composition comprising an RSV Glycoprotein fragment of claim 1- or a polynucleotide encoding the fragment to a subject before, during, or after the administration of an immunogenic composition, such that the subjects immune response is enhanced.

83-86. (canceled)

87. A method for identifying a candidate compound that modulates an immune response in a subject, the method comprising:
a) providing a cell expressing an RSV Glycoprotein nucleic acid molecule;
(b) contacting the cell with a candidate compound; and
(c) comparing the expression of the nucleic acid molecule in the cell contacted with the candidate compound with the expression of the nucleic acid molecule in a control cell not contacted with the candidate compound, wherein an alteration in the expression identifies the candidate compound as a candidate compound that modulates an immune response.

88. A method for identifying a candidate compound that modulates an immune response in a subject, the method comprising:
(a) providing a cell expressing a RSV Glycoprotein;
(b) contacting the cell with a candidate compound; and
(c) comparing the biological activity of the RSV Glycoprotein in the cell contacted with the candidate compound to a control cell not contacted with the candidate compound, wherein an alteration in the biological activity of the RSV Glycoprotein identifies the candidate compound as a candidate compound that modulates an immune response in a subject.

89-93. (canceled)

94. A method for identifying a candidate compound that modulates an immune response in a subject, the method comprising:
a) contacting a RSV Glycoprotein with a candidate compound; and
(b) detecting binding of the candidate compound to the RSV Glycoprotein, wherein the binding identifies the candidate compound as a candidate compound that modulates an immune response in a subject.

95. A method for enhancing an immunomodulatory activity of an RSV Glycoprotein, the method comprising:
a) introducing an alteration in a naturally occurring RSV Glycoprotein amino acid sequence; and
b) detecting an alteration in the immunomodulatory activity of the RSV Glycoprotein.

96-103. (canceled)

104. A method for selecting an RSV Glycoprotein nucleic acid molecule having improved immunomodulatory activity, the method comprising:
a) introducing an alteration in a naturally occurring RSV Glycoprotein nucleic acid sequence; and
b) detecting an alteration in the immunomodulatory activity of the encoded RSV Glycoprotein.

105. A method for treating or preventing an influenza viral infection in a subject, the method comprising administering to the subject a polypeptide comprising at least a fragment of an RSV Glycoprotein or a nucleic acid molecule encoding said polypeptide to the subject.

106. A method for treating or preventing malaria in a subject, the method comprising administering to the subject a polypeptide comprising at least a fragment of an RSV Glycoprotein or a nucleic acid molecule encoding the polypeptide to the subject.

107-109. (canceled)
